SEQUENCE LISTING (84 sequences)

SEQ ID NO: 1 - protein, 402 aa, Mus musculus
MADKILRAKRKQFINSVSIGTINGLLDELLEKRVLNQEEMDKIKLANITAMDKARDLCDHVSKKGPQASQIFITYICNEDCYLAGILELQSAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 2 - protein, 402 aa, synthetic construct
  REGION 1..402: note = CASP1_C285G
MADKILRAKRKQFINSVSIGTINGLLDELLEKRVLNQEEMDKIKLANITAMDKARDLCDHVSKKGPQASQIFITYICNEDCYLAGILELQSAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQAGRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 3 - protein, 326 aa, synthetic construct
  REGION 1..326: note = CASP1_RV
MVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGHLLVCDVPIRAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKD

SEQ ID NO: 4 - protein, 317 aa, synthetic construct
  REGION 1..317: note = CASP1_RV2
MSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGHLLVCDVPIRAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKD

SEQ ID NO: 5 - protein, 88 aa, synthetic construct
  REGION 1..88: note = Casp1 S, p10 (AA315-402)
GIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 6 - protein, 27 aa, synthetic construct
APSAETFVATEDSKGGHPSSSETKEEQ

SEQ ID NO: 7 - protein, 9 aa, synthetic construct
LLVCDVPIR

SEQ ID NO: 8 - protein, 536 aa, synthetic construct
  REGION 1..536: note = iCASP1
MGSRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLESGGGSAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 9 - protein, 5 aa, synthetic construct
  REGION 1..5: note = SGGGS linker
SGGGS

SEQ ID NO: 10 - protein, 220 aa, synthetic construct
  REGION 1..220: note = F36V-FKBP
MGSRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKVDSSRDRNKPFKFMLGKQEVIRGWEEGVAQMSVGQRAKLTISPDYAYGATGHPGIIPPHATLVFDVELLKLE

SEQ ID NO: 11 - protein, 217 aa, synthetic construct
  REGION 1..217: note = F36M-FKBP
MGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLE

SEQ ID NO: 12 - protein, 533 aa, synthetic construct
  REGION 1..533: note = dCASP1
MGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLESGGGSAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKDSVRDSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGH

SEQ ID NO: 13 - protein, 236 aa, synthetic construct
  REGION 1..236: note = dasher GFP
MTALTEGAKLFEKEIPYITELEGDVEGMKFIIKGEGTGDATTGTIKAKYICTTGDLPVPWATLVSTLSYGVQCFAKYPSHIKDFFKSAMPEGYTQERTISFEGDGVYKTRAMVTYERGSIYNRVTLTGENFKKDGHILRKNVAFQCPPSILYILPDTVNNGIRVEFNQAYDIEGVTEKLVTKCSQMNRPLAGSAAVHIPRYHHITYHTKLSKDRDERRDHMCLVEVVKAVDLDTYQ

SEQ ID NO: 14 - protein, 276 aa, synthetic construct
MPSAFEKVVKNVIKEVSGSRGDLIPVDSLRNSTSFRPYCLLNRKFSSSRFWKPRYSCVNLSIKDILEPSAPEPEPECFGSFKVSDVVDGNIQGRVMLSGMGEGKISGGAAVSDSSSASMNVCILRVTQKTWETMQHERHLQQPENKILQQLRSRGDDLFVVTEVLQTKEEVQITEVHSQEGSGQFTLPGALCLKGEGKGHQSRKKMVTIPAGSILAFRVAQLLIGSKWDILLVSDEKQRTFEPSSGDRKAVGQRHHGLNVLAALCSIGKQLSLLSD

SEQ ID NO: 15 - protein, 284 aa, synthetic construct
  REGION 1..284: note = GSDMD_NTER_FLAG
MPSAFEKVVKNVIKEVSGSRGDLIPVDSLRNSTSFRPYCLLNRKFSSSRFWKPRYSCVNLSIKDILEPSAPEPEPECFGSFKVSDVVDGNIQGRVMLSGMGEGKISGGAAVSDSSSASMNVCILRVTQKTWETMQHERHLQQPENKILQQLRSRGDDLFVVTEVLQTKEEVQITEVHSQEGSGQFTLPGALCLKGEGKGHQSRKKMVTIPAGSILAFRVAQLLIGSKWDILLVSDEKQRTFEPSSGDRKAVGQRHHGLNVLAALCSIGKQLSLLSDDYKDDDDK

SEQ ID NO: 16 - protein, 472 aa, synthetic construct
  REGION 1..472: note = MLKL_FLAG_MT
MDKLGQIIKLGQLIYEQCEKMKYCRKQCQRLGNRVHGLLQPLQRLQAQGKKNLPDDITAALGRFDEVLKEANQQIEKFSKKSHIWKFVSVGNDKILFHEVNEKLRDVWEELLLLLQVYHWNTVSDVSQPASWQQEDRQDAEEDGNENMKVILMQLQISVEEINKTLKQCSLKPTQEIPQDLQIKEIPKEHLGPPWTKLKTSKMSTIYRGEYHRSPVTIKVFNNPQAESVGIVRFTFNDEIKTMKKFDSPNILRIFGICIDQTVKPPEFSIVMEYCELGTLRELLDREKDLTMSVRSLLVLRAARGLYRLHHSETLHRNISSSSFLVAGGYQVKLAGFELSKTANSISRTAKSTKAERSSSTIYVSPERLKNPFCLYDIKAEIYSFGIVLWEIATGKIPFEGCDSKKIRELVAEDKKQEPVGQDCPELLREIINECRAHEPSQRPSVDGILERLSAVEESTDKKVDYKDDDDK

SEQ ID NO: 17 - protein, 173 aa, Mus musculus
MNNRWILHAAFLLCFSTTALSVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 18 - protein, 182 aa, synthetic construct
MNNRWILHAAFLLCFSTTALSINYKQLQLQERTNIRKCQELLEQLNGKINLTYRADFKIPMEMTEKMQKSYTAFAIQEMLQNVFLVFRNNFSSTGWNETIVVRLLDELHQQTVFLKTVLEEKQEERLTWEMSSTALHLKSYYWRVQRYLKLMKYNSYAWMVVRAEIFRNFLIIRRLTRNFQN

SEQ ID NO: 19 - protein, 292 aa, synthetic construct
  REGION 1..292: note = Ddx58_FLAG_RIGI_NTER
MDYKDDDDKTAEQRQNLQAFRDYIKKILDPTYILSYMSSWLEDEEVQYIQAEKNNKGPMEAASLFLQYLLKLQSEGWFQAFLDALYHAGYCGLCEAIESWDFQKIEKLEEHRLLLRRLEPEFKATVDPNDILSELSECLINQECEEIRQIRDTKGRMAGAEKMAECLIRSDKENWPKVLQLALEKDNSKFSELWIVDKGFKRAESKADEDDGAEASSIQIFIQEEPECQNLSQNPGPPSEASSNNLHSPLKPRNYQLELALPAKKGKNTIICAPTGCGKTFVSLLICEHHLK

SEQ ID NO: 20 - protein, 41 aa, synthetic construct
  REGION 1..41: note = Reps1 non relevant protein
MKKVVVNGRVLELFRAAQLANDVVLQIMELCGATRLGYFGR

SEQ ID NO: 21 - protein, 269 aa, Mus musculus
MATVPELNCEMPPFDSDENDLFFEVDGPQKMKGCFQTFDLGCPDESIQLQISQQHINKSFRQAVSLIVAVEKLWQLPVSFPWTFQDEDMSTFFSFIFEEEPILCDSWDDDDNLLVCDVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 22 - protein, 173 aa, Mus musculus; identical to SEQ ID NO: 17

SEQ ID NO: 23 - protein, 189 aa, synthetic construct
  REGION 1..189: note = polyepitope vaccine
MAEAGQSLVISASIIVFNLLELEGDYRDDHIFSLYFMAAAKVVVNGRVLELFRAAQLANDVVLQIMELCGATRLGAAADIPTGIPVHLELASMTNMELMSSIVHQQVFPTVASAAAGISSAESLKISQAVHAAHAEINEAGREVVGSAEAGAAALLPDEVSGLEQLESIINFEKLTEWTSSNVMEERKI

SEQ ID NO: 24 - protein, 141 aa, synthetic construct
MWLQNLLFLGIVVYSLSAPTRSPITVTRPWKHVEAIKEALNLLDDMPVTLNEEVEVVSNEFSFKKLTCVQTRLKIFEQGLRGNFTKLKGALNMTASYYQTYCPPTPETDCETQVTTYADFIDSLKTFLTDIPFECKKPGQK

SEQ ID NO: 25 - protein, 35 aa, synthetic construct
  REGION 1..35: note = Dpagt1 tumor specific adjuvant
EAGQSLVISASIIVFNLLELEGDYRDDHIFSLYFM

SEQ ID NO: 26 - protein, 35 aa, synthetic construct
  REGION 1..35: note = Reps1 tumor specific adjuvant
KVVVNGRVLELFRAAQLANDVVLQIMELCGATRLG

SEQ ID NO: 27 - protein, 35 aa, synthetic construct
  REGION 1..35: note = Adpgk tumor-specific adjuvant
DIPTGIPVHLELASMTNMELMSSIVHQQVFPTVAS

SEQ ID NO: 28 - protein, 35 aa, Gallus gallus
GISSAESLKISQAVHAAHAEINEAGREVVGSAEAG

SEQ ID NO: 29 - protein, 35 aa, Gallus gallus
LLPDEVSGLEQLESIINFEKLTEWTSSNVMEERKI

SEQ ID NO: 30 - skipped

SEQ ID NO: 31 - protein, 312 aa, synthetic construct
MGSTARTGHFVDQHRQALIARVTEVDGVLDALHGSVLTEGQYQAVRAETTSQDKMRKLFSFVPSWNLTCKDSLLQALKEIHPYLVMDLEQSGGGGSGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLETRGVQVETISPGDGRTFPKRGQTCVVHYTGMLEDGKKMDSSRDRNKPFKFMLGKQEVIRGWEEGVAQMSVGQRAKLTISPDYAYGATGHPGIIPPHATLVFDVELLKLE

SEQ ID NO: 32 - protein, 495 aa, synthetic construct
  REGION 1..495: note = GSDMD_WT_FLAG
MPSAFEKVVKNVIKEVSGSRGDLIPVDSLRNSTSFRPYCLLNRKFSSSRFWKPRYSCVNLSIKDILEPSAPEPEPECFGSFKVSDVVDGNIQGRVMLSGMGEGKISGGAAVSDSSSASMNVCILRVTQKTWETMQHERHLQQPENKILQQLRSRGDDLFVVTEVLQTKEEVQITEVHSQEGSGQFTLPGALCLKGEGKGHQSRKKMVTIPAGSILAFRVAQLLIGSKWDILLVSDEKQRTFEPSSGDRKAVGQRHHGLNVLAALCSIGKQLSLLSDGIDEEELIEAADFQGLYAEVKACSSELESLEMELRQQILVNIGKILQDQPSMEALEASLGQGLCSGGQVEPLDGPAGCILECLVLDSGELVPELAAPIFYLLGALAVLSETQQQLLAKALETTVLSKQLELVKHVLEQSTPWQEQSSVSLPTVLLGDCWDEKNPTWVLLEECGLRLQVESPQVHWEPTSLIPTSALYASLFLLSSLGQKPCDYKDDDDK

SEQ ID NO: 33 - protein, 173 aa, synthetic construct
  REGION 1..173: note = IL1B_CALRSP
MGLLSVPLLLGLLGLAAADPAVPIRQLHYRLRDEQQKSLVLSDPYELKALHLNGQNINQQVIFSMSFVQGEPSNDKIPVALGLKGKNLYLSCVMKDGTPTLQLESVDPKQYPKKKMEKRFVFNKIEVKSKVEFESAEFPNWYISTSQAEHKPVFLGNNSGQDIIDFTMESVSS

SEQ ID NO: 34 - protein, 178 aa, Mus musculus
MNNRWILHAAFLLCFSTTALSNFGRLHCTTAVIRNINDQVLFVDKRQPVFEDMTDIDQSASEPQTRLIIYMYKDSEVRGLAVTLSVKDSKMSTLSCKNKIISFEEMDPPENIDDIQSDLIFFQKRVPGHNKMEFESSLYEGHFLACQKEDDAFKLILKKKDENGDKSVMFTLTNLHQS

SEQ ID NO: 35 - protein, 318 aa, synthetic construct
  REGION 1..318: note = hCASP1_RV2
MGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGHAYVHDAPVRDQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

SEQ ID NO: 36 - protein, 174 aa, Homo sapiens
MTNKCLLQIALLLCFSTTALSAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 37 - protein, 269 aa, Homo sapiens
MAEVPKLASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 38 - protein, 303 aa, synthetic construct
  REGION 1..303: note = CASP1_RV2_NTR
MGIKKAHIEKDFIAFCSSTPDNVSWRHPVRGSLFIESLIKHMKEYAWSCDLEDIFRKVRFSFEQPEFRLQMPTADRVTLTKRFYLFPGHLLVCDVPIRAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKD

SEQ ID NO: 39 - protein, 317 aa, synthetic construct
  REGION 1..317: note = CASP1_RV2_C305G
MSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGHLLVCDVPIRAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQAGRGEKQGVVLLKD

SEQ ID NO: 40 - protein, 317 aa, synthetic construct
  REGION 1..317: note = CASP1_RV2_D108A
MSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGHLLVCAVPIRAPSAETFVATEDSKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKD

SEQ ID NO: 41 - protein, 317 aa, synthetic construct
  REGION 1..317: note = CASP1_RV2_D124A
MSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGHLLVCDVPIRAPSAETFVATEASKGGHPSSSETKEEQNKEDGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKD

SEQ ID NO: 42 - protein, 317 aa, synthetic construct
  REGION 1..317: note = CASP1_RV2_D143A
MSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGHLLVCDVPIRAPSAETFVATEDSKGGHPSSSETKEEQNKEAGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKD

SEQ ID NO: 43 - protein, 317 aa, synthetic construct
  REGION 1..317: note = CASP1_RV2_D108A_D124A_D143A
MSEEDFLTDAIFEDDGIKKAHIEKDFIAFCSSTPDNVSWRHPVRGSLFIESLIKHMKEYAWSCDLEDIFRKVRFSFEQPEFRLQMPTADRVTLTKRFYLFPGHLLVCAVPIRAPSAETFVATEASKGGHPSSSETKEEQNKEAGTFPGLTGTLKFCPLEKAQKLWKENPSEIYPIMNTTTRTRLALIICNTEFQHLSPRVGAQVDLREMKLLLEDLGYTVKVKENLTALEMVKEVKEFAACPEHKTSDSTFLVFMSHGIQEGICGTTYSNEVSDILKVDTIFQMMNTLKCPSLKDKPKVIIIQACRGEKQGVVLLKD

SEQ ID NO: 44 - protein, 318 aa, synthetic construct
  REGION 1..318: note = hCASP1_RV2_C306G
MGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGHAYVHDAPVRDQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQAGRGDSPGVVWFKD

SEQ ID NO: 45 - protein, 318 aa, synthetic construct
  REGION 1..318: note = hCASP1_RV2_D108A
MGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGHAYVHAAPVRDQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

SEQ ID NO: 46 - protein, 318 aa, synthetic construct
  REGION 1..318: note = hCASP1_RV2_D140A
MGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGHAYVHDAPVRDQTSGNYLNMQDSQGVLSSFPAPQAVQANPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

SEQ ID NO: 47 - protein, 318 aa, synthetic construct
  REGION 1..318: note = hCASP1_RV2_D124A
MGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGHAYVHDAPVRDQTSGNYLNMQASQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

SEQ ID NO: 48 - protein, 310 aa, synthetic construct
  REGION 1..310: note = hCASP1_RV2_NTR2
MEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGHAYVHDAPVRDQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

SEQ ID NO: 49 - protein, 304 aa, synthetic construct
  REGION 1..304: note = hCASP1_RV2_NTR
MAIKKAHIEKDFIAFCSSTPDNVSWRHPTMGSVFIGRLIEHMQEYACSCDVEEIFRKVRFSFEQPDGRAQMPTTERVTLTRCFYLFPGHAYVHDAPVRDQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

SEQ ID NO: 50 - protein, 276 aa, synthetic construct
  REGION 1..276: note = hCASP1_RV2_NTR_GSL
MAIKKAHIEKDFIAFCSSTPDNVSWRHPTMGSVFIGRLIEHMQEYACSCDVEEIFRKVRFSFEQPDGRAQMPTTERVTLTRCFYLFPGHGSGSGSGSGNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

SEQ ID NO: 51 - protein, 276 aa, synthetic construct
  REGION 1..276: note = hCASP1_RV2_NTR_CCS
MAIKKAHIEKDFIAFCSSTPDNVSWRHPTMGSVFIGRLIEHMQEYACSCDVEEIFRKVRFSFEQPDGRAQMPTTERVTLTRCFYLFPGHAYVHDAPVRNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

SEQ ID NO: 52 - protein, 404 aa, Homo sapiens
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 53 - protein, 492 aa, synthetic construct
  REGION 1..492: note = hGSDMD_WT_FLAG
MGSAFERVVRRVVQELDHGGEFIPVTSLQSSTGFQPYCLVVRKPSSSWFWKPRYKCVNLSIKDILEPDAAEPDVQRGRSFHFYDAMDGQIQGSVELAAPGQAKIAGGAAVSDSSSTSMNVYSLSVDPNTWQTLLHERHLRQPEHKVLQQLRSRGDNVYVVTEVLQTQKEVEVTRTHKREGSGRFSLPGATCLQGEGQGHLSQKKTVTIPSGSTLAFRVAQLVIDSDLDVLLFPDKKQRTFQPPATGHKRSTSEGAWPQLPSGLSMMRCLHNFLTDGVPAEGAFTEDFQGLRAEVETISKELELLDRELCQLLLEGLEGVLRDQLALRALEEALEQGQSLGPVEPLDGPAGAVLECLVLSSGMLVPELAIPVVYLLGALTMLSETQHKLLAEALESQTLLGPLELVGSLLEQSAPWQERSTMSLPPGLLGNSWGEGAPAWVLLDECGLELGEDTPHVCWEPQAQGRMCALYASLALLSGLSQEPHDYKDDDDK

SEQ ID NO: 54 - protein, 92 aa, synthetic construct
  REGION 1..92: note = hCASP1_CARD
MADKVLKEKRKLFIRSMGEGTINGLLDELLQTRVLNKEEMEKVKRENATVMDKTRALIDSVIPKGAQACQICITYICEEDSYLAGTLGLSAD

SEQ ID NO: 55 - protein, 27 aa, synthetic construct
  REGION 1..27: note = hCASP1_CDL
QTSGNYLNMQDSQGVLSSFPAPQAVQD

SEQ ID NO: 56 - protein, 178 aa, synthetic construct
  REGION 1..178: note = hCASP1_p20
NPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKD

SEQ ID NO: 57 - protein, 19 aa, synthetic construct
  REGION 1..19: note = hCASP1_IDL
SVGVSGNLSLPTTEEFEDD

SEQ ID NO: 58 - protein, 88 aa, synthetic construct
  REGION 1..88: note = hCASP1_p10
AIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 59 - protein, 17 aa, synthetic construct
  REGION 1..17: note = binding sequence
GGVADLIKKFESISKEE

SEQ ID NO: 60 - protein, 14 aa, synthetic construct
  REGION 1..14: note = IDL sequence
SEEDFLTDAIFEDD

SEQ ID NO: 61 - protein, 14 aa, synthetic construct
  REGION 1..14: note = inter-domain linker
GNLSLPTTEEFEDD

SEQ ID NO: 62 - protein, 15 aa, synthetic construct
  REGION 1..15: note = linker
GGGGSGGGGSGGGGS

SEQ ID NO: 63 - protein, 35 aa, synthetic construct
  REGION 1..35: note = linker
GGGGSGGGGSGGGGSGGGGSGGGGSGGGGSGGGGS

SEQ ID NO: 64 - protein, 15 aa, synthetic construct
  REGION 1..15: note = linker
GGGSSGGGSSGGGSS

SEQ ID NO: 65 - protein, 8 aa, synthetic construct
SIINFEKL

SEQ ID NO: 66 - protein, 9 aa, synthetic construct
  REGION 1..9: note = aa
AYVHDAPVR

SEQ ID NO: 67 - protein, 9 aa, synthetic construct
  REGION 1..9: note = linker
GSGSGSGSG

SEQ ID NO: 68 - protein, 60 aa, synthetic construct
FPAPQAVQDNPAMPTSSGSEGNVKLCSLEEAQRIWKQKSAEIYPIMDKSSRTRLALIICN

SEQ ID NO: 69 - protein, 59 aa, synthetic construct
SSETKEEQNKEDGTFPGLTGTLKFCPLEKAQKLWKENPSEIYPIMNTTTRTRLALIICN

SEQ ID NO: 70 - protein, 60 aa, synthetic construct
PNKKAHPNMEAGPPESGESTDALKLCPHEEFLRLCKERAEEIYPIKERNNRTRLALIICN

SEQ ID NO: 71 - protein, 56 aa, synthetic construct
HHGEANLEMEEPEESLNTLKLCSPEEFTRLCREKTQEIYPIKEANGRTRKALIICN

SEQ ID NO: 72 - protein, 60 aa, synthetic construct
TSVKPLLQIEAGPPESAESTNILKLCPREEFLRLCKKNHDEIYPIKKREDRRRLALIICN

SEQ ID NO: 73 - protein, 60 aa, synthetic construct
AHESHLMLTAPHGLQSSEVQDTLKLCPRDQFCKIKTERAKEIYPVMEKEGRTRLALIICN

SEQ ID NO: 74 - protein, 57 aa, synthetic construct
VFIGRLIEHMQEYACSCDVEEIFRKVRFSFEQPDGRAQMPTTERVTLTRCFYLFPGH

SEQ ID NO: 75 - protein, 57 aa, synthetic construct
LFIESLIKHMKEYAWSCDLEDIFRKVRFSFEQPEFRLQMPTADRVTLTKRFYLFPGH

SEQ ID NO: 76 - protein, 57 aa, synthetic construct
IFITQLITCFQKYSWCCHLEEVFRKVQQSFETPRAKAQMPTIERLSMTRYFYLFPGN

SEQ ID NO: 77 - protein, 57 aa, synthetic construct
YFITRLISCFRKHACSCHLFDIFLKVQQSFEKASIHSQMPTIDRATLTRYFYLFPGN

SEQ ID NO: 78 - protein, 57 aa, synthetic construct
IFITELITCFQKYSCCCHLMEIFRKVQKSFEVPQAKAQMPTIERATLTRDFYLFPGN

SEQ ID NO: 79 - protein, 57 aa, synthetic construct
LFISKLIDCFKKYCWCYHLEEIFRKVQHSFEVPGELTQMPTIERVSMTRYFYLFPGN

SEQ ID NO: 80 - protein, 14 aa, synthetic construct
  REGION 1..14: note = CASP1_RV2
GNLSLPTTEEFEDD

SEQ ID NO: 81 - protein, 19 aa, synthetic construct
  REGION 1..19: note = CASP1_RV2
SVGVSGNLSLPTTEEFEDD

SEQ ID NO: 82 - protein, 14 aa, synthetic construct
  REGION 1..14: note = CASP1_RV2
SEEDFLTDAIFEDD

SEQ ID NO: 83 - protein, 24 aa, synthetic construct
MVLLKDSVRDSEEDFLTDAIFEDD

SEQ ID NO: 84 - protein, 15 aa, synthetic construct
MSEEDFLTDAIFEDD

(19) United States
(12) Patent Application Publication
(10) Pub. No.: US 2024/0325526 A1
(43) Pub. Date: Oct. 3, 2024

(54) IMMUNE-STIMULATORY COMPOSITIONS AND USE THEREOF

(71) Applicant: Nykode Therapeutics ASA, Oslo (NO)

(72) Inventors: Jeroen VAN BERGEN, Leiden (NL); Gerben Carolus Martinus ZONDAG, Leiden (NL)

(21) Appl. No.: 18/751,098

(22) Filed: Jun. 21, 2024

Related U.S. Application Data
(63) Continuation of application No. 17/600,164, filed on Sep. 30, 2021.

(30) Foreign Application Priority Data
Apr. 2, 2019 (EP) 19166877.1

(51) Int. Cl. A61K 39/39; A61K 39/00

(52) U.S. Cl. CPC A61K 39/39; A61K 2039/55516; A61K 2039/55527

(57) ABSTRACT

The invention relates to a constitutively active pro-inflammatory caspase, comprising shuffled p10 and p20 domains, for use in a method of stimulating an immune response in an individual. The invention further relates to an immune-stimulating composition, comprising said constitutively active pro-inflammatory caspase, comprising shuffled p10 and p20 domains and a pharmacologically acceptable excipient, and its use in a method of treating an individual.

### CROSS REFERENCE TO RELATED APPLICATIONS

This application is a continuation of U.S. patent application Ser. No. 17/600,164, filed Sep. 30, 2021, titled “IMMUNE-STIMULATORY COMPOSITIONS AND USE THEREOF,” now U.S. Patent Application Publication No. 2022/0211843, which is a national phase application under 35 U.S.C. 371 of International Patent Application No. PCT/NL2020/050225, filed Apr. 2, 2020, titled “IMMUNE-STIMULATORY COMPOSITIONS AND USE THEREOF,” now International Publication No. WO/2020/204714.

### SEQUENCE LISTING

The instant application contains a Sequence Listing which has been submitted via Patent Center and is hereby incorporated by reference in its entirety. Said.xml copy, created on Jun. 6, 2024 is named 14834-702300 and is 106,276 bytes in size.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The invention relates generally to immune stimulatory compositions comprising an inducer of pyroptotic cell death such as a constitutively active pro-inflammatory caspase, comprising swapped p10 and p20 domains.

### INTRODUCTION

Effective immune responses against cancer and pathogens require the activation of T cells specific for protein fragments (antigens) selectively expressed by malignantly transformed or infected cells. Upon activation in draining lymph nodes, these activated T cells re-enter the circulation and invade the affected tissues to clear aberrant—but not healthy—cells. After this first encounter with antigen, an expanded subset of these T cells persists and is more easily activated. These memory T cells thus reduce the susceptibility to re-infection with the same or a similar pathogen and may similarly reduce a risk of cancer recurrence. Antigen specificity and memory are both features characteristic of the adaptive immune system.

To prevent infections, individuals can be immunised by administration of antigen preparations, either derived from the infectious pathogen itself or produced synthetically in the form of peptide, protein, mRNA or DNA. This procedure, otherwise known as vaccination, may also be applicable to the prevention and treatment of cancer. Innate immune system activation is an absolute requirement for the induction of adaptive immune responses. Critical to vaccine efficacy, particularly in the case of synthetic vaccines, is therefore the inclusion of an adjuvant that activates cells of the innate immune system (McKee and Marrack, 2017. Curr Opin Immunol 47:44-51).

The most commonly used adjuvant is alum, which is composed of aluminum salts. More recently developed adjuvants tend to mimic pathogen-associated molecular patterns (PAMPS) to target pattern recognition receptors (PRR) on innate immune cells (Kanzler et al., 2007. Nat Med 13:552-9; Wu, 2016. Immunology 148:315-25; Vasou et al., 2017. Viruses 9: pii: E186). These adjuvants generally work well with protein and peptide vaccines, but not with genetic vaccines consisting of RNA or DNA (Li and Petrovsky, 2016. Expert Rev Vaccines 15:313-29).

While the first vaccine derived from an infectious pathogen was made in the 18th century, genetic vaccines are much younger. The first DNA vaccines were developed towards the end of the 20th century, and the first report on mRNA vaccines dates from 2004 (Carralot et al., 2004. Cell Mol Life Sci 61:2418-24). Although many genetic vaccines are currently in clinical trials, none have yet been approved for human use. A major challenge in this field is the discovery of effective adjuvants. Such adjuvants are usually combined with the mRNA or DNA and encode immune stimulatory proteins such as cytokines, chemokines, and more recently components of PRR signaling pathways. Of these adjuvants, only two have thus far shown promise in human clinical trials: cytokines IL-12 and GM-CSF (CSF2) (Li et al., 2017. Clin Vaccine Immunol 24: c00263-17; Richie et al., 2012. Hum Vaccin Immunother 8:1564-84).

There is thus a need to provide efficient inducers of innate immune responses that can be used as adjuvants for genetic vaccines, i.e. nucleic acid-based vaccines.

### BRIEF DESCRIPTION OF THE INVENTION

The invention provides a constitutively active pro-inflammatory caspase for use in a method of stimulating an immune response in an individual, preferably a T-cell mediated immune response, comprising administering said constitutively active pro-inflammatory caspase to the individual.

Infected or transformed cells can alert the immune system by the way they die. Apoptosis tends to be immunologically silent, as apoptotic cells do not emit inflammatory signals and are swiftly cleared by macrophages. In contrast, cells undergoing inflammatory cell death induce immune activation. Pyroptosis and necroptosis are recently discovered forms of programmed necrosis that lead to the release of damage-associated molecular patterns (DAMPS; Wallach et al., 2016. Science 352:51-58). These DAMPS include intracellular molecules that perform non-inflammatory functions in living cells (e.g. ATP, high-mobility group box 1 (HMGB1)), and cytokines such as IL-1β, IL-18 and IL-33. Pyroptotic and necroptotic DAMPS require membrane disruption to be released into the extracellular milieu, where they can bind pattern recognition receptors (PRRs) and/or cytokine receptors to activate innate immune cells.

Cells undergoing necroptosis are known to elicit anti-tumour immunity. Injection of necroptotic cells into mice activates IFNγ-, TNF- and IL-2-producing T cells specific for the tumour antigens contained within these cells. These T cells are cytotoxic and can efficiently eliminate tumour cells (Yatim et al., 2015. Science 350:328-334; Aaes et al., 2016. Cell Rep 15:274-278). Furthermore, forced expression of a necroptotic effector molecule, mixed lineage kinase domain-like (MLKL) in tumour cells activates tumour-specific T cells that help clear that tumour (van Hoecke et al., 2018. Nat Commun 9:3417). The importance of necroptosis in anti-tumour immunity is also underscored by the fact that expression of receptor interacting protein kinase 3 (RIPK3), a key player in this pathway just upstream of MLKL, is frequently reduced in tumour cells. Active MLKL forms pores in the cell membrane, upon which the cells ‘explode’ due to osmosis, leading to abundant DAMP release. Thus, necroptosis can stimulate anti-tumour immunity by triggering a cytotoxic response characterised by the pro-inflammatory cytokines IFNγ and TNF, a type of immune response typically employed for anti-viral immunity.

Pyroptosis on the other hand occurs mainly in macrophages exposed to bacteria such as Salmonella, fungi, and some viruses. Specific to pyroptosis are the release of pro-inflammatory cytokines IL18 and IL1β, and the formation of gasdermin D (GSDMD) pores in the cell membrane (Amarante-Mendes et al., 2018. Front Immunol 9:2379-97). GSDMD induces a morphologically unique type cell death characterised by the formation of pyroptotic bodies in the absence of osmotically induced cell swelling and bursting (Chen et al., 2016. Cell Res 26:1007-20). Cytokine production and cell death both rely on the activation of pro-inflammatory caspases, most notably caspase-1. This pathway can be detrimental to combating viral infections, as mice lacking caspase-1 are less susceptible to influenza infection (Ren et al., 2017. Sci Rep 7:7625) and inhibition of caspase-1 prolonged survival of mice after infection with rabies virus (Koraka et al., 2018. Vaccine 10.1016/j.vaccine.2018.04.002). Thus, in contrast with necroptosis, pyroptosis emerges as an anti-microbial response in a limited subset of cells (macrophages), with a different signature cytokine profile (IL18, IL1β) and morphology. It is therefore not obvious to target this pathway for stimulating an immune response against viruses or cancer.

Several documents, including US 2014/037685, US 2018/311343, WO 2018/049014 and WO 2018/106753 have suggested that an inducer of pyroptosis may stimulate an immune response. This inducer of pyroptosis preferably is or encodes a protein selected from an apoptosis-associated speck protein containing a CARD (ASC), an inflammatory caspase such as caspase-1, a gasdermin such as gasdermin-D or gasdermin E, and/or a variant of any one of these proteins. However, none of these documents describes and shows a constitutively active pro-inflammatory caspase generated by domain swapping.

A constitutively active pro-inflammatory caspase according to the invention, preferably human constitutively active pro-inflammatory caspase, comprises swapped p20 and p10 domains, optionally connected by a protease cleavable site. Said constitutively active pro-inflammatory caspase according to the invention preferably is a constitutively active pro-inflammatory caspase-1. Said constitutively active pro-inflammatory caspase according to the invention preferably lacks a caspase-recruitment domain (CARD).

A constitutively active pro-inflammatory caspase, preferably human caspase-1, according to the invention preferably comprises a glycine corresponding to G401 (SEQ ID NO: 1), which is located at a distance up to 40 amino acids residues from a cysteine corresponding to C135 (SEQ ID NO: 1), preferably at a distance of less than 10 amino acid residues such as 0-2 amino acid residues. A preferred constitutively active pro-inflammatory caspase according to the invention preferably comprise a glycine corresponding to G403 (SEQ ID NO: 52), which is located at a distance from 0 to 40 amino acids residues from a cysteine corresponding to C136 (SEQ ID NO:52), preferably at a distance of 0-10 amino acid residues such as 0-2 amino acid residues. This effectively removes or replaces up to 16 N-terminal amino acids of the p20 domain and up to 1 C-terminal amino acid of the p10 domain.

A constitutively active pro-inflammatory caspase according to the invention preferably lacks a p20-p10 interdomain linker (IDL) at the N-terminal part of the protein. Hence, the region N-terminal to the caspase p10 domain, a remnant of the IDL, is preferably shorter than 15 amino acids, preferably shorter than 7 amino acids, more preferably shorter than 2 amino acids, most preferably absent.

A preferred immune response is directed against a tumour or infection that is present in an individual or is induced to prevent occurrence or recurrence of a tumour or infection in an individual. Administration of said inducer of pyroptosis, preferably said constitutively active pro-inflammatory caspase comprising shuffled p10 and p20 domains, thus is for prophylactic and/or therapeutic administration.

In an embodiment, said inducer of pyroptosis, preferably said constitutively active pro-inflammatory caspase comprising shuffled p10 and p20 domains, is administered into a tumour of the individual, preferably by intra-tumoural injection.

In an embodiment, said inducer of pyroptosis, preferably said constitutively active pro-inflammatory caspase comprising shuffled p10 and p20 domains, is administered topically and/or systemically as an adjuvant of a vaccine.

Said inducer of pyroptosis, preferably said constitutively active pro-inflammatory caspase comprising shuffled p10 and p20 domains, preferably is administered in combination with one or more accessory molecules such as an immune checkpoint inhibitor and/or a further immune stimulating molecule such as a chemokine or a cytokine, preferably one or more accessory molecules as exemplified in Table 3. A preferred accessory molecule is an immune checkpoint inhibitor such as antibodies against PD1 or its ligands, antibodies against CTLA-4, and/or a cytokine such as Interleukin-12 (IL12) and/or granulocyte-macrophage colony-stimulating factor (CSF2).

In an embodiment, said inducer of pyroptosis, preferably said constitutively active pro-inflammatory caspase comprising shuffled p10 and p20 domains, is provided as an expression molecule, preferably expressing an inducer of pyroptosis as listed in any one of Tables 1-2, preferably said constitutively active pro-inflammatory caspase comprising shuffled p10 and p20 domains.

The invention further provides an immune-stimulating composition, comprising an inducer of pyroptosis, preferably said constitutively active pro-inflammatory caspase comprising shuffled p10 and p20 domains, and a pharmacologically acceptable excipient.

Said immune-stimulating composition according to the invention preferably further comprises at least one antigen or antigen-encoding nucleic acid molecule. Said immune-stimulating composition may additionally comprise comprising an accessory immune stimulating molecule such as an immune checkpoint inhibitor and/or a. further immune stimulating molecule such as a cytokine and/or a chemokine. A preferred accessory molecule is or comprises one or more of the molecules provided in Table 3.

Said inducer of pyroptosis, preferably said constitutively active pro-inflammatory caspase comprising shuffled p10 and p20 domains, in an immune-stimulating composition according to the invention preferably is selected from the molecules depicted in any one of Tables 1-2.

The invention further provides an immune-stimulating composition according to the invention, for use in a method of treating an individual suffering from a tumour or an infection.

The invention further provides a method of stimulating an immune response in an individual, preferably a T-cell mediated immune response, comprising providing a composition of the invention and administering said composition to the individual.

### BRIEF DESCRIPTION OF THE DRAWINGS

FIG. 1. Caspase-1 constructs. The CASP1_WT (SEQ ID NO: 1) construct codes for the entire wild-type sequence of Mus musculus Casp1, while in CASP1_C285G (SEQ ID NO: 2) the cysteine (at position 284) in the active site of Casp1 is replaced with a Glycine, rendering this site enzymatically inactive. In CASP1_RV (SEQ ID NO: 3) and CASP1_RV2 (SEQ ID NO: 4), the C-terminal p10 domain (SEQ ID NO: 5) is moved to the N-terminus and separated from the CARD-p20 linker sequence (SEQ ID NO: 6) by the Caspase-1 cleavage site of Il1b (SEQ ID NO: 7), which is indicated by a downward arrow. CASP1_RV retains a small part of p20 (5 C-terminal amino acids) at the N-terminus, which has been removed in CASP1_RV2. Finally, in iCASP1 (SEQ ID NO: 8) a CARD-less version of Caspase-1 is connected by a small SGGGS linker (SEQ ID NO: 9) to an AP1903-inducible dimerisation domain from FKBP (F36V-FKBP, SEQ ID NO: 10). F36M-FKBP (SEQ ID NO: 11) in dCASP1 (SEQ ID NO: 12) induces spontaneous dimerisation.

FIG. 2. CASP1_RV and CASP1_RV2 induce cell death. B16F10 cells were transfected with 25 ng of the indicated plasmids (SEQ ID NO: NO 14-20) mixed with 25 ng GFP-plasmid (SEQ ID NO: 13, labeled p53). Two days after transfection, cells were harvested and stained with 7-AAD, a marker of dead cells. (A) Caspase-1 constructs. (B) control constructs. An expression vector coding for a non-relevant small peptide Reps1 (SEQ ID NO: 20) served as negative control, and GSDMD_NTER (SEQ ID NO: 14) as a positive control. Note that a construct representing the N-terminus of DExD/H-Box Helicase 58 (Ddx58; FLAG_RIGI_NTER, SEQ ID NO: 19) also induces cell death.

FIG. 3. CASP1_RV2 induces pro-IL-1β processing and IL-1β secretion. B16F10 cells were transfected with 0.6 ng of the indicated plasmids mixed with 10 ng empty vector, pro-IL1β (SEQ ID NO: 21, IL1B_FL, caspase-1 dependent), or mature IL-1β (SEQ ID NO: 22, IL1B_WT, caspase-1 independent). When indicated, cells were treated with 10 nM AP1903 one day after transfection. Two days after transfection, supernatants were harvested for IL-1β ELISA and measurement of LDH release. Note that under these experimental conditions, none of the transfections resulted in cell death, as they did not cause significant LDH release (data not shown).

FIG. 4. CASP1_RV2 improves T cell responses and anti-tumour immunity. C57BL/6 mice were vaccinated intradermally with polyepitope vaccine (SEQ ID NO: 23, 10 μg) together with empty vector control, CASP1_RV2 (SEQ ID NO: 4) or reference adjuvant CSF2 (SEQ ID NO: 24, 10 μg) on day 0. On days 6 (A), 9 (B), 13 (C), and 44 (D) post vaccination, OVA-specific CD8 T cells responses were evaluated by tetramer staining. Mice were challenged with B16-OVA cells on day 29, and tumour outgrowth was then followed for another 3 months. E. Tumour-free survival.

FIG. 5. Active forms of Ddx58 (RIG-I) and Gsdmdc1 (GSDMD) do not improve CD8 T cell immunity. C57BL/6 mice were vaccinated intradermally with 10 μg polyepitope vaccine (closed symbols) or empty vector (open symbols), together with 10 μg empty vector control (−) or plasmids encoding active forms of Ddx58 (SEQ ID NO: 19, FLAG_RIGI_NTER), Caspase-1 (SEQ ID NO: 4, CASP1_RV2), Gsdmdc1 (SEQ ID NO: 14, GSDMD_NTER), or Csf2 (SEQ ID NO: 24, CSF2). On days 8 post vaccination, OVA-specific CD8 T cell responses were evaluated by tetramer staining.

FIG. 6. Progressive N-terminal truncations of constitutively active mouse caspase-1 reveal requirement for retaining part of the p20-p10 inter-domain linker (IDL) at the N-terminus for optimal CASP1_RV activity. (A) Schematic representation of N-terminal variants of CASP1_RV. The N-terminal sequences upstream of the p10 domain are MVLLKDSVRDSEEDFLTDAIFEDD (CASP1_RV, SEQ ID NO: 83), MSEEDFLTDAIFEDD (CASP1_RV2, SEQ ID NO: 84) and M (CASP1_RV2_NTR, SEQ ID NO: 38). B16-F10 cells were co-transfected with 0.5 or 10 ng/well (100 μl) of the indicated caspase-1 (or GSDMD N-terminus, SEQ ID NO: 14) and 10 ng/well IL-1β DNA plasmids, and (B) IL-1β (0.5 ng/well caspase-1) and (C) LDH activity (0.5 and 10 ng/well caspase-1) were measured in supernatants 2 days later.

FIG. 7. Constitutively active mouse caspase-1 (CASP1_RV2) is approximately 30-fold more potent than wild-type caspase-1 (CASP1_WT). B16-F10 cells were co-transfected with the indicated amounts of caspase-1 plasmids (serially diluted in insert-less plasmids to maintain a constant total amount of plasmid in the assay) together with 10 ng pro-IL1β plasmid per 100 μl, and (A) IL-1β and (B) LDH activity in supernatants were assessed 2 days later. Active site mutants of CASP1_RV2 [SEQ ID NO: 4] and CASP1_WT [SEQ ID NO: 1] were CASP1_RV2_C305G [SEQ ID NO: 39] and CASP1_C285G [SEQ ID NO: 2],respectively.

FIG. 8. 293 cells were co-transfected with the indicated concentrations of human caspase-1 (hCASP1) variants [SEQ ID NOs: 35, 44, 49-52], serially diluted in empty vector plasmid to keep the total DNA concentration constant, together with 10 ng/well plasmid either encoding (A) pro-IL1β [SEQ ID NO: 37] or (B) human GSDMD [SEQ ID NO: 53]. Two days later supernatants were harvested to determine (A) IL1β concentrations or (B) LDH activity.

FIG. 9. (A) Crystal structure of human caspase-1. Note the proximity of the C-terminally located Gly403 of the p10 domain (amino acids 317-404) and the N-terminal Leu135 of the p20 domain (amino acid 135-297). Adapted from Yang et al. (2018) Proc. Natl. Acad. Sci. U.S.A. 115:6792-6797. (B) (SEQ ID NO: 68-79) Sequence alignment of human and mouse pro-inflammatory caspases. Note the conservation of the mCasp1-C135/hCASP1-C136 (next to hCASP1-L135) and mCasp1-G401/hCASP1-G403 amino acid residues.

### DETAILED DESCRIPTION OF THE INVENTION

### 4.1 Definitions

The term “cell death”, as is used herein, is the event of a biological cell ceasing to carry out its functions. This may be the result of the natural process, or may result from factors such as disease, localized injury, or death of an organism encompassing the cells. Different types of cell death, including apoptosis and pyroptosis, are often defined by morphological criteria.

The term “programmed cell death”, as is used herein, refers to any type of cell death engaged by an active predestined molecular mechanism.

The term “apoptosis”, as is used herein, refers to cell death accompanied by rounding-up of the cell, retraction of pseudopodes, reduction of cellular volume (pyknosis), chromatin condensation, nuclear fragmentation (karyorrhexis), and plasma membrane blebbing.

The term “necrosis”, as is used herein, refers to unprogrammed cell death by cellular damage or infiltration by pathogens. Necrosis is characterized by a gain in cell volume, swelling of organelles, rupture of the plasma membrane and subsequent loss of intracellular contents.

The term “necroptosis”, as is used herein, refers to a programmed form of cell death by a caspase-independent fashion, involving activation of mixed lineage kinase domain like pseudokinase (MLKL) and the acute permeabilization of the plasma membrane. Necroptosis can serve as a anti-viral defense mechanism, allowing the cell to undergo “cellular suicide” in the presence of viral caspase inhibitors, thereby restricting virus replication.

The term “pyroptosis”, as is used herein, refers to a programmed form of cell death in which activation of inflammatory caspases leads to cleavage of gasdermin and permeabilization of the cell membrane. In addition, the activated caspases may cleave pro-cytokines such as pro-Interleukin 1 beta (proIL1β) and pro-IL18 into their biologically active forms, which are then released as a result of cell permeabilization. Pyroptosis occurs upon infection with intracellular pathogens. Pyroptosis promotes the rapid clearance of various bacterial and viral infections by removing intracellular replication niches and enhancing the host's defensive responses.

The term “T-cell mediated immune response”, as is used herein, refers to protective mechanisms that are responsible for detecting and destroying intracellular pathogens, e.g., cells that are infected with viruses or bacteria. T-cell mediated immune responses can also contribute to the destruction of tumour cells. Key players are CD4+ and CD8+ T cells, which produce inflammatory cytokines such as Interferon gamma (IFN-γ) and Tumor Necrosis Factor (TNF). In addition, CD8+ T cells have the ability to induce apoptosis of infected and/or transformed cells.

The term “apoptosis-associated speck-like protein containing a CARD (ASC)”, as is used herein, refers to an adapter protein (human protein UniProt: Q9ULZ3) that is s composed of two protein-protein interaction domains: a N-terminal PYRIN-PAAD-DAPIN domain (PYD) and a C-terminal caspase-recruitment domain (CARD). The human gene encoding ASC is termed PYCARD (HGNC: 16608; Entrez Gene: 29108; Ensembl: ENSG00000103490). Activated ASC is a key mediator in pyroptosis and serves as a scaffold for activation of inflammatory caspases such as caspase-1.

The term “caspase-1”, as is used herein, refers to a protein (human protein UniProt: P29466) that is a member of the cysteine-aspartic acid protease (caspase) family. Caspases exist as inactive proenzymes that undergo proteolytic processing at conserved aspartic residues to produce 2 subunits, large and small, that dimerize to form the active enzyme. The human gene encoding caspase-1 is termed CASP1 (HGNC: 1499; Entrez Gene: 834; Ensembl: ENSG00000137752.

The term “gasdermin D”, as is used herein, refers to a protein that is cleaved by an inflammatory caspase into a N-terminal and C-terminal part. After cleavage, the N-terminal part moves to the plasma membrane where it forms pores, thus promoting release of mature interleukin (IL) 1B and IL18 and triggering pyroptosis. Full length gasdermin D comprises 484 amino acids (human protein UniProt: P57764), of which amino acid residues 1-275 constitute the N-terminal part, and amino acid residues 276-484 the C-terminal part. The human gene encoding gasdermin D is termed GSDMD (HGNC: 25697; Entrez Gene: 79792; Ensembl: ENSG00000104518).

The term “inflammatory caspase”, as is used herein, refers to a caspase that is able to induce pyroptosis. Said inflammatory caspase preferably is selected from one or more of caspase-1, caspase-4, caspase-5, and caspase-12.

The term ‘protein variant” as is used herein, refers to a protein that has a similar activity as the endogenous protein. A protein variant can be an active part of a protein, or a homologous but not identical protein or part thereof. Said homologous protein or part thereof is active and preferably more than 70% identical to the corresponding human protein, more preferred at least 80% identical, such as more than 90% identical, more than 95% identical or more than 99% identical to the corresponding human protein.

As will be understood by a person skilled in the art, the term “% identity” refers to the % identity as determined over the full length of the proteins, except when the protein variant refers to an active part of a protein.

A preferred protein variant is an active or inducible protein. Preferred protein variants of ASC, caspase 1, and gasdermin D are depicted in Tables 1 and 2.

The term “active or inducible protein”, as is used herein, refers to a protein that is dominantly active, without a need for activation, or of which the activity can be induced, for example by overexpression, transcriptional activation or by dimerization. An example of inducible transcriptional activation is provided by a tetracycline-controlled gene expression system (Yamada et al., 2018. Cell Rep 25:487-500.e6). An example of inducible dimerization is provided by an optical dimerizer system that is based on the interacting domains of phytochromes and cryptochromes of bacteria and plants. Examples of such interacting domains are Arabidopsis thaliana cryptochrome 2 (CRY2) and CRY2-interacting basic Helix-Loop-Helix (CIB1; Taslimi et al., 2016. Nature Chem Biol 12:425-430), and a FK506-binding protein (FKBP) and FKBP-rapamycin binding (Frb) domain of mTOR, which can be induced to dimerize by rapamycin (Kohler and Bertozzi, 2003. Chem Biol 10:1303-11) and variants thereof such as Shield 1 (Banaszynski et al., 2006. Cell 126:995-1004). Commercially available systems include iDimerize Inducible Homodimer System (Takara, Kusatsu, Japan).

The term “antigen”, as is used herein, refers to a molecule that can be specifically recognised by the adaptive immune system, that is, by B cells and/or T cells. A sequence within an antigen that is bound by an antibody or a T-cell receptor is called an epitope. A preferred antigen comprises one or more epitopes specific for or highly expressed in cancer, including neo-epitopes, epitopes from pathogens such as bacteria and viruses, and/or synthetic epitopes that do not occur in nature. The term “neo-epitope”, as is used herein, refers to an epitope that arises through non-synonymous somatic DNA mutations that change the amino acid coding sequences. A preferred T cell epitope comprises 8-20 amino acid residues, more preferred 8-13 amino acid residues. A preferred antigen is or comprises a polyepitope, comprising 2-50, preferably 5-25 individual epitopes, preferably each contained within a sequence of 8-40 amino acid residues. The individual epitopes in a polyepitope may be alternated by spacer sequences of, preferably, 1-10 amino acid residues.

Said antigen preferably also comprises G-actin, or F-actin binding sequences such as GGVADLIKKFESISKEE (SEQ ID NO: 59) (Riedl et al 2008, Nat Methods. 5:605). Actin is liberated by dying cells and binds to Dendritic cell Natural killer lectin Group Receptor 1 (DNGR-1) on conventional type 1 DCs (cDC1s; Ahrens et al. 2012, Immunity 36:635-645; Zhang et al. 2012. Immunity 36:646-657), important for cross-priming CD8 T cell responses (Schulz et al. 2018, Cell Reports 24:419-428). The inclusion of actin or actin-binding sequences may therefore result in more efficient cross-priming of CD8 T cells by cDC1s and thus a more effectively stimulated immune response.

The term “accessory molecule”, as is used herein, refers to a molecule that may facilitate T-cell mediated immune responses, including an immune checkpoint inhibitor and a further immune stimulating molecule such as a chemokine and/or a cytokine.

The term “immune checkpoint inhibitor”, as is used herein, refers to a molecule that blocks an inhibitory interaction between immune cells and other cells or cytokines and which may thereby increase the killing of cancer cells. Examples of checkpoint interacting molecules are PD-1/PD-L1 and CTLA-4/B7-1/B7-2. A preferred immune checkpoint inhibitor is a molecule that blocks an interaction between PD-1 and PD-L1. Said molecule that blocks an interaction between PD-1 and PD-L1 preferably is an antibody against PD1 and/or an antibody against PDL1.

The term “a further immune stimulating molecule”, as is used herein, refers to a molecule that facilitates T-cell mediated immune responses such as cytotoxic T lymphocyte induction. Such molecules include pro-inflammatory cytokines, for example interleukins (IL-) such as IL-1β, IL-6, IL-12, granulocyte-macrophage colony stimulating factor (CSF2), and tumor necrosis factor (TNF), chemokines such as monocyte chemoattractant protein or MCP-1, as are listed in Table 3. A preferred another immune stimulating molecule is a an IL-12 family member, such as IL-12, IL-23, IL-27 and/or IL-35 and/or CSF2.

The term “genetic vaccine”, or gene vaccine, as is used herein, refers to a vaccine that comprises one or more RNA or DNA nucleic acid sequences that encode antigens against which an immune response is to be directed. Genetic vaccines that induce cellular immune responses provide a means to generate specific cellular responses, while avoiding risks associated with, for example, attenuated pathogenic bacteria or viruses.

The term “vaccine”, as is used herein, refers to an immune-stimulating molecule, preferably an antigen, that stimulates an immune response against said molecule. Said immune response preferably confers active immunity against an agent that comprises and/or expresses said immune-stimulating molecule by stimulating the immune system to attack the agent. The term vaccine includes a composition comprising an immune-stimulating molecule and an adjuvant.

The term “systemic administration”, as is used herein, refers to parenteral administration such as, for example, intravenous, intraperitoneal, intranasal, intradermal, transdermal or intramuscular administration.

The term “local administration”, as is used herein, refers to topical administration to body surfaces such as, for example, administration to the skin, eyes, mucous membranes, or through inhalation.

The term “shuffled”, also termed “swapped”, as used herein, refers to a recombinant protein in which the order of conserved domains has been altered. A caspase comprising shuffled or swapped p10 and p20 domains is a recombinant caspase in which the p10 domain is N-terminal to the p20 domain.

### 4.2 A Protein as an Inducer of Pyroptosis

In one embodiment, an inducer of pyroptosis, preferably said constitutively active pro-inflammatory caspase comprising shuffled p10 and p20 domains, optionally connected by a protease cleavable site, is provided as a protein that is expressed in a host cell. Said constitutively active pro-inflammatory caspase preferably is a constitutively active pro-inflammatory caspase-1.

Based on the human caspase-1 sequences (UniProt accession code P29466), caspase-1 is generated as a propeptide. Human caspase-1 (SEQ ID NO:52) contains a caspase recruitment domain (CARD; SEQ ID NO:54) from amino acid residue 1 to amino acid residue 92. Residues 93-119 constitute a linker between the CARD and p20 domains, also known as the CARD-domain linker (CDL; SEQ ID NO:55). The p20 subunit domain (SEQ ID NO:56) stretches from amino acid residue 120 to amino acid residue 297 and contains an enzymatically active site around Cys285. The p20 domain is followed by a small p20-p10 inter-domain linker (IDL; SEQ ID NO: 57) from amino acid residue 298 to amino acid residue 316, and a p10 subunit (SEQ ID NO: 58) from amino acid residue 317 to amino acid residue 404.

It has been suggested that a CARD domain is required in active caspase-possessing an N-terminal CARD domain (Boucher et al., 2018. J Exp Med 215:827-840). Surprisingly, a preferred constitutively active pro-inflammatory caspase according to the invention comprising shuffled p10 and p20 domains preferably lacks a caspase-recruitment domain (CARD).

Said constitutively active pro-inflammatory caspase, was based on constitutively active versions of human apoptosis-inducing executioner caspase-3 and -6 (Srinivasula et al., 1998. J Biol Chem 273:10107-11). As described in this article, for example in the design of active caspase-3, the N-terminal p20 and C-terminal p10 domains were swapped and separated by a short (8 AA) caspase-3 cleavage site. A part of the sequence upstream of p10, including a few p20 amino acids, was also moved by this swap. Hence, the N-terminus of resulting active caspase began with four p20 amino acids, followed by the small p20-p10 inter-domain linker (IDL) and p10 (Srinivasula et al., 1998. J Biol Chem 273:10107-11).

It is noted that enzymatically inactive pro-caspase-3 already is a stable dimer that changes conformation upon cleavage of the inter-domain linker (IDL), suggesting a spring-loaded mechanism where cutting the IDL releases strain and allows the enzyme to reorganize into the active conformation. In contrast, caspase-1 requires cleavage of the IDL and dimerisation for activation. Hence, it was investigated whether a constitutively active caspase-1 could be generated by shuffling of the p10 and p20 domains.

A constitutively active murine inflammatory caspase-1 (CASP1_RV, SEQ ID NO: 3) was generated as described in Srinivasula et al., 1998 (Srinivasula et al., 1998. J Biol Chem 273:10107-11). Accordingly, the N-terminus of murine CASP1_RV began with five p20 amino acids and the IDL. However, testing three CASP1_RV variants progressively lacking more of this N-terminus showed that removal of the N-terminal p20 remnant in CASP1_RV2 [SEQ ID NO: 4] increased, but further removal of the IDL in CASP1_RV2_NTR [SEQ ID NO: 38] decreased the activity of CASP1_RV. In other words, the most active CASP1_RV2 variant retained most of the highly negatively charged IDL sequence SEEDFLTDAIFEDD (SEQ ID NO: 60) at its N-terminus. Compared to wild-type murine caspase-1 (CASP1_WT), CASP1_RV2 was approximately 30-fold more potent in in vitro assays. Thus, despite differences between human caspase-3 and mouse caspase-1, such as the presence of a CARD domain in caspase-1 and a difference in activation mechanism, the Srinivasula-approach indeed generated constitutively active mouse caspase-1.

The design of a constitutively active human caspase-1 variant (hCASP1_RV2, SEQ ID NO: 35) was based on murine CASP1_RV2, and therefore also began with 14 amino acids of the inter-domain linker GNLSLPTTEEFEDD (SEQ ID NO: 61). Surprisingly, and in striking contrast with murine CASP1_RV2, removing all N-terminal residues upstream of p10 (hCASP1_RV2_NTR, SEQ ID NO: 49) greatly increased its activity.

A preferred constitutively active pro-inflammatory caspase comprising shuffled p10 and p20 domains is thus a constitutively active pro-inflammatory caspase-1 comprising shuffled p10 and p20 domains which lacks one or more of the negatively charged amino acid residues D and E in the IDL sequence in front of the P10 domain, preferably lacks the inter-domain linker SVGVSGNLSLPTTEEFEDD (SEQ ID NO: 57), preferably the complete inter-domain linker SVGVSGNLSLPTTEEFEDD (SEQ ID NO: 57).

In a preferred constitutively active pro-inflammatory caspase comprising shuffled p10 and p20 domains, a start codon, ATG, preferably is positioned in front of the p10 sequences, preferably directly in front of the p10 sequences.

The intervening sequence between shuffled p10 and p20 domains appears to be flexible. Crystal structures of caspase-1 in its active conformation (Yang et al., 2018. Proc Natl Acad Sci USA 115:6792-6797) seem to suggest that the distance between the C-terminus of the p10 domain and the N-terminus of the p20 domain is small. Removing aspartic acid protease sites in this region of mouse and human caspase-1 comprising shuffled p10 and p20 domains [SEQ ID NO:s 40-43, 45-47] did not affect their ability to process pro-IL1β, suggesting that autoproteolysis did not contribute to its activity (data not shown). Rather, this region appeared to serve merely as a flexible linker. In fact, reducing the size of the linker to 9 amino acids was not a neutral event, as was expected, but surprisingly increased the activity of hCASP1_NTR, irrespective of the presence of an autoproteolytic target site in the smaller linker. Compared to wild-type human caspase-1, the resulting hCASP1_NTR_GSL [SEQ ID NO: 50] was approximately 30-fold more potent in in vitro assays. Thus, and again in contrast to mouse caspase-1, swapping the p10 and p20 domains did not significantly increase the activity of human caspase-1. However, removing the IDL-remnant at the N-terminus and reducing the size of a linker between the swapped p10 and p20 domains unexpectedly did yield a constitutively active human caspase-1.

A preferred constitutively active pro-inflammatory caspase comprising shuffled p10 and p20 domains comprises a glycine corresponding to G401 (SEQ ID NO:1) which is located at a distance from 0 to 40 amino acids residues from a cysteine corresponding to C135 (SEQ ID NO: 1), preferably at a distance of 1-10 amino acid residues. A most preferred constitutively active pro-inflammatory caspase according to the invention has a reduced distance between G401 and C135 residues and therefore lacks a linker between p10 and p20.

SEQ ID NO: 1 corresponds to the mouse caspase-1 sequence. A skilled person will understand that the term “corresponding to”, as is used herein above, is meant to indicate that the equivalent amino acid residue in a different caspase-1 sequence, might not be the amino acid residue at the same position in different caspase. For example, mouse C135 corresponds to human C136. Similarly, G401 in the mouse corresponds to human G403. Hence, a preferred constitutively human active pro-inflammatory caspase-1 comprising shuffled p10 and p20 domains comprises a glycine corresponding to G403 which is located at a distance from 0 to 40 amino acids residues from a cysteine corresponding to C136, preferably at a distance of 0-10 amino acid residues. A most preferred constitutively active human pro-inflammatory caspase-1 according to the invention has a reduced distance between G403 and C136 residues and therefore lacks a linker between shuffled p10 and p20 domains.

Said linker may be a linker polypeptide comprising from about 1 amino acid residue to about 40 amino acid residues, most preferred to about 35 amino acid residues such as to 30 amino acid residues, to 20 amino acid residues, to 15 amino acid residues, to 10 amino acid residues, such as 2 amino acid residues. Some preferred examples of such linker polypeptide sequences include Gly-Ser linkers, for example of the type (Glyx Sery) z such as, for example, (Gly4 Ser) 3 (SEQ ID NO: 62), (Gly4 Ser) 7 (SEQ ID NO: 63) or (Gly3 Ser2) 3 (SEQ ID NO: 64), as described in WO 99/42077, and the GS30, GS15, GS9 and GS7 linkers described in, for example, WO 06/040153 and WO 06/122825.

Commonly used expression systems for heterologous protein production include E. coli, baculovirus, yeast, Chinese Hamster Ovary cells (CHO), human embryonic kidney (HEK) cells and derivatives thereof including HEK293 cells including HEK293T, HEK293E, HEK-293F and HEK-293 FT (Creative Biolabs, NY, USA), and PER.C6® cells (Thermo Fisher Scientific, MA, USA). The efficiency of expression of recombinant proteins in heterologous systems depends on many factors, both on the transcriptional level and the translational level.

Said inducer of pyroptosis, for example a protein selected from ASC, caspase-1, and/or gasdermin D, or a variant thereof, preferably an active or inducible variant, more preferably said constitutively active pro-inflammatory caspase comprising shuffled p10 and p20 domains, may be produced using prokaryotic cells, preferably E. coli, fungi, most preferably filamentous fungi or yeasts such as Saccharomyces cerevisiae and Pichia pastoris, or eukaryotic cells, preferably mammalian cells such as HEK cells and derivatives thereof. Commercial systems for expression in mammalian cells are available, for example the Expi293 mammalian transient protein expression system (Thermo Fisher Scientific, Waltham (MA) USA).

Production of an inducer of pyroptosis in filamentous fungi is preferably performed as described by Joosten et al., 2005. J Biotechnol 120:347-359, which is included herein by reference.

Production of an inducer of pyroptosis in Pichia pastoris is preferably performed as described by Rahbarizadch et al., 2006. J Mol Immunol 43:426-435, which is included herein by reference.

Production of an inducer of pyroptosis in HEK cells and/or derivatives thereof is preferably performed as described by Thomas and Smart, 2005. J Pharmacol Toxicol Methods 51:187-200, and/or Lin et al., 2015. PLOS ONE 10: e0123562.

Said inducer of pyroptosis, preferably said constitutively active pro-inflammatory caspase comprising shuffled p10 and p20 domains, is preferably produced by the provision of a nucleic acid encoding said inducer of pyroptosis to a cell of interest. Said nucleic acid, preferably DNA, is preferably produced by recombinant technologies, including the use of polymerases, restriction enzymes, and ligases, as is known to a skilled person. Alternatively, said nucleic acid is provided by artificial gene synthesis, for example by synthesis of partially or completely overlapping oligonucleotides, or by a combination of organic chemistry and recombinant technologies, as is known to the skilled person. Said nucleic acid is preferably codon-optimised to enhance expression of the inducer of pyroptosis in the selected cell or cell line. Further optimization preferably includes removal of cryptic splice sites, removal of cryptic polyA tails and/or removal of sequences that lead to unfavourable folding of the mRNA. The presence of an intron flanked by splice sites may encourage export from the nucleus in eukaryotic cells. In addition, the nucleic acid preferably encodes a protein export signal for secretion of the inducer of pyroptosis out of the cell into the periplasm of prokaryotes or into the growth medium, allowing efficient purification of the inducer of pyroptosis.

Methods for purification of an inducer of pyroptosis are known in the art and are generally based on chromatography, such as ion exchange, to remove contaminants. In addition to contaminants, it may also be necessary to remove undesirable derivatives of the product itself such as degradation products and aggregates. Suitable purification process steps are provided in Berthold and Walter, 1994 (Berthold and Walter, 1994. Biologicals 22:135-150).

As an alternative, or in addition, a recombinant inducer of pyroptosis may be tagged with a specific tag by genetic engineering to allow the protein attach to a column specific to the tag and therefore be isolated from impurities. The purified protein is then exchanged from the affinity column with a decoupling reagent. The method has been increasingly applied for purifying recombinant protein. Conventional tags for proteins, such as histidine tag, is used with an affinity column that specifically captures the tag (eg., a Ni-IDA column for a histidine tag) to isolate the protein from other impurities. The protein is then exchanged from the column using a decoupling reagent according to the specific tag (eg., immidazole for histidine tag). This method is more specific, when compared with traditional purification methods. Suitable further tags include c-myc domain, hemagglutinin tag, and maltose-binding protein, glutathione-S-transferase, maltose-binding protein, FLAG tag peptide, biotin acceptor peptide, streptavidin-binding peptide and calmodulin-binding peptide, as presented in Chatterjee, 2006. Cur Opin Biotech 17, 353-358). Methods for employing these tags are known in the art and may be used for purifying the inducer of pyroptosis.

### 4.3 An Expression Construct as an Inducer of Pyroptosis

Further provided is an expression construct that encodes an inducer of pyroptosis, preferably said constitutively active pro-inflammatory caspase comprising shuffled p10 and p20 domains, according to the invention. Said expression construct preferably comprises means for high expression levels such as strong promoters, for example of viral origin (e.g., human cytomegalovirus) or promoters derived from genes that are highly expressed in a cell such as a mammalian cell (Running Deer and Allison, 2004. Biotechnol Prog 20:880-889; U.S. Pat. No. 5,888,809). The construct preferably comprises a selection system such as, for example, expression of glutamine synthetase or expression of dihydrofolate reductase for amplification of the vector in a suitable recipient cell, as is known to the skilled person.

Said construct may be a viral vector, preferably a viral vector that is able to transduce dividing and non-dividing cancer cells. Said viral vector preferably is a recombinant adeno-associated viral vector, a herpes simplex virus-based vector, a pox virus-based vector such as a modified vaccinia Ankara-based vector as described in WO2011128704, or a lentivirus-based vector such as a human immunodeficiency virus-based vector. Said viral vector most preferably is a retroviral-based vector such as a lentivirus-based vector such as a human immunodeficiency virus-based vector, or a gamma-retrovirus-based vector such as a vector based on Moloney Murine Leukemia Virus (MoMLV), Spleen-Focus Forming Virus (SFFV), Myeloproliferative Sarcoma Virus (MPSV) or on Murine Stem Cell Virus (MSCV). A preferred retroviral vector is the SFG gamma retroviral vector (Rivière et al., 1995. PNAS 92:6733-6737).

Retroviruses, including a gamma-retrovirus-based vector, can be packaged in a suitable complementing cell that provides Group Antigens polyprotein (Gag)-Polymerase (Pol) and/or Envelop (Env) proteins. Suitable packaging cells are human embryonic kidney derived 293T cells, Phoenix cells (Swift et al., 2001. Curr Protoc Immunol, Chapter 10: Unit 10 17C), PG13 cells (Loew et al., 2010. Gene Therapy 17:272-280) and Flp293A cells (Schucht et al., 2006. Mol Ther 14:285-92).

A preferred expression construct is a non-viral expression construct for in vivo expression of an inducer of pyroptosis, preferably said constitutively active pro-inflammatory caspase comprising shuffled p10 and p20 domains. Non-viral vectors include circular or linear DNA molecules, and RNA molecules such as messenger RNA. A non-viral expression construct may be packaged in liposomes, lipoplexes or polyplexes, and/or provided as a molecular conjugate. Minicircle DNA molecules or linear DNA molecules free of plasmid bacterial DNA sequences may be generated in vitro and may express an inducer of pyroptosis at high levels in vivo.

Said expression construct may further comprise a nucleic acid encoding another immune stimulating molecule such as a cytokine.

As an alternative, or in addition, said expression construct may be combined with an expression construct coding for another immune stimulating molecule.

### 4.4 Application of Induced Pyroptosis

The invention provides an inducer of pyroptosis, preferably said constitutively active pro-inflammatory caspase comprising shuffled p10 and p20 domains, for use in a method of stimulating an immune response in an individual, preferably a T-cell mediated immune response, comprising administering said inducer of pyroptosis to the individual. Said inducer of pyroptosis, preferably said constitutively active pro-inflammatory caspase comprising shuffled p10 and p20 domains, is preferably used for preventive or therapeutic administration in humans suffering from a disease, or at risk to suffer from a disease. Said diseases include but are not limited to measles, rubella, cholera, meningococcal disease, influenza, diphtheria, mumps, tetanus, hepatitis A, hepatitis B, hepatitis E, pertussis, tuberculosis, pneumoccocal disease, typhoid fever, poliomyelitis, tick-borne encephalitis, haemophilus influenzae type b, rabies, varicella and herpes zoster (shingles), human papilloma-virus, human immunodeficiency virus, respiratory syncytial virus, cytomegalovirus, rotavirus gastroenteritis, yellow fever, Japanese encephalitis, malaria, dengue fever, Zika virus-related microcephaly, anthrax, plague, Q fever, smallpox, or from a non-infectious diseases such as cancer.

In one embodiment, said disease is a cancer. Said individual may suffer or may be treated to prevent the recurrence of a cancer, including but not limited to a carcinoma, an adenoma, a melanoma, a sarcoma, a leukemia, a germ cell cancer, a blastoma, and/or a lymphoma.

Said carcinoma includes adenocarcinoma, squamous cell carcinoma, adenosquamous carcinoma, anaplastic carcinoma, large cell carcinoma and small cell carcinoma and encompasses bladder cancer, breast cancer, kidney cancer, pancreatic cancer, ovarian cancer, lung cancer, liver cancer, head and neck cancer, squamous cell carcinoma, colorectal cancer, cervical cancer, renal cell carcinoma, stomach cancer, prostate cancer, melanoma, brain cancer, thyroid cancer, uterine cancer, esophageal cancer.

Said sarcoma includes Askin's tumour, chondrosarcoma, Ewing's sarcoma, malignant schwannoma, osteosarcoma and soft tissue sarcomas, including fibrosarcoma, leiomyosarcoma, liposarcoma, and rhabdomyosarcoma.

Said leukemia includes acute and chronic leukemia, comprising lymphoblastic leukemia such as Burkitt's leukemia, myelogenous leukemia such as acute myelogenous leukemia (AML) and chronic myelogenous leukemia (CML), Hairy cell leukemia (HCL), T-cell prolymphocytic leukemia, large granular lymphocytic leukemia, adult T-cell leukemia, and clonal eosinophilia.

Said germ cancer includes germinomatous or seminomatous germ-cell tumours such as germinoma, dysgerminoma and seminoma, and nongerminomatous or nonseminomatous germ-cell tumours such as teratoma and polyembryoma.

Said blastoma includes hepatoblastoma, medulloblastoma, nephroblastoma, neuroblastoma, pancreatoblastoma, pleuropulmonary blastoma, retinoblastoma, glioblastoma multiforme, and gonadoblastoma.

Said lymphoma includes Hodgkin's lymphoma, non-Hodgkin's lymphoma, B-cell lymphoma, T-cell lymphoma, Burkitt's lymphoma, follicular lymphoma, mantle cell lymphoma, small lymphocytic lymphoma, and lymphoplasmacytic lymphoma.

### 4.5 Intratumoural Administration

Preferred cancers in embodiments of the invention are cancers that form a body mass, such as sarcomas, germ cancers and carcinomas. An inducer of pyroptosis, for example an active or inducible protein selected from ASC, caspase-1, and/or gasdermin D, more preferably said constitutively active pro-inflammatory caspase comprising shuffled p10 and p20 domains, may be administered locally to an individual that is suffering from a cancer, preferably intratumourally. The resulting pyroptosis of the cancer cells will release damage-associated molecular patterns together with cancer-specific antigens, preferably cancer neoepitopes, that will activate tumour-specific T cells that will start attacking the remaining cancer cells and eventually even may clear the cancer.

Said inducer of pyroptosis, for example an active or inducible protein selected from ASC, caspase-1, and/or gasdermin D, more preferably said constitutively active pro-inflammatory caspase comprising shuffled p10 and p20 domains, may be intratumourally administered as a protein or, preferably, as an expression construct that expresses said inducer of pyroptosis in the cancer cells. Said administration of an inducer of pyroptosis into a tumour preferably is by intratumoural injection, preferably by injection or electroporation, as is known to a person skilled in the art. Said electroporation may be applied on isolated cells in vitro or, preferably, in vivo. An effective amount of an inducer of pyroptosis, preferably said constitutively active pro-inflammatory caspase comprising shuffled p10 and p20 domains, as a protein that is administered intratumourally is a dosage large enough to produce the desired effect in which the symptoms of the cancer are ameliorated or even nullified. A therapeutically effective amount preferably does not cause adverse side effects. Generally, a therapeutically effective amount may vary with the individual's age, condition, and sex, as well as the extent of the disease and can be determined by one of skill in the art. The dosage may be adjusted by the individual physician in the event of any complication. A therapeutically effective amount may vary from about 1 microgram to about 100 milligram, preferably from about 10 microgram to about 10 milligram, most preferably from about 0.1 milligram to about 1 milligram, in one or repeated dose administrations, for one or more days.

Suitable transfection reagents for transducing cells such as cancer cells with a protein include Saint-PROTEIN® transfection reagent (Synvolux, Leiden, the Netherlands).

Said effective amount of an inducer of pyroptosis, preferably said constitutively active pro-inflammatory caspase comprising shuffled p10 and p20 domains, as a protein preferably is provide as a buffered solution, preferably having a pH values between 5 and 9, preferably between 6 and 8. Said buffered solution may, for example, comprise a phosphate-, histidine-or succinate-based buffer, polysorbate, trehalose dihydrate,and/or methionine.

An expression construct such as a non-viral expression construct, expressing an inducer of pyroptosis, preferably said constitutively active pro-inflammatory caspase comprising shuffled p10 and p20 domains, may be administered in an effective amount to an individual in need thereof. Preferred is repeated administration, such as repeated administration on 2 to 5 or more consecutive days in order to effectively induce an immune response against the cancer in order to treat a cancer.

An expression construct such as a non-viral expression construct, expressing an inducer of pyroptosis, preferably said constitutively active pro-inflammatory caspase comprising shuffled p10 and p20 domains, preferably is administered by injection or electroporation into a tumour. Preparations for intratumoural administration may comprise sterile aqueous or non-aqueous solutions suspensions, and emulsions. Examples of non-aqueous solvents are propylene glycol, polyethylene glycol, vegetable oils such as olive oil, and injectable organic esters such as ethyl oleate. Preparations for intratumoural administration preferably comprise aqueous carriers such as water optionally including a buffering agent and salts, saline such as sodium chloride solution, Ringer's dextrose, dextrose and sodium chloride, or lactated Ringer's. Preservatives and other additives may also be present such as, for example, antimicrobials, anti-oxidants, chelating agents, and inert gases and the like.

A therapeutically effective amount of an expression construct expressing an inducer of pyroptosis, such as a non-viral expression construct, may vary from about 0.001 mg to about 100 mg, preferably from about 0.01 mg to about 10 mg, most preferably from about 0.1 mg to about 1 mg.

The invention further provides a method of ameliorating and/or treating an individual suffering from a cancer, and/or preventing recurrence of a cancer, the method comprising administering an effective amount of an inducer of pyroptosis, preferably said constitutively active pro-inflammatory caspase comprising shuffled p10 and p20 domains, to said individual to stimulate an immune response in the individual against a cancer neoepitope, thereby ameliorating and/or treating said individual.

Suitable transfection reagents for transfecting or transducing cells, especially cancer cells, with an expression construct such as a non-viral expression construct include cationic polyplexes such as polyethylenimine, liposomes or lipoplexes comprising cationic lipids such as DOTAP or pyridinium-based lipids such as Saint-DNA and Saint-mRNA transfection reagents (Synvolux, Leiden, the Netherlands).

### 4.6 Systemic Administration

The invention provides an inducer of pyroptosis, preferably said constitutively active pro-inflammatory caspase comprising shuffled p10 and p20 domains, for use in a method of stimulating an immune response in an individual, preferably a T-cell mediated immune response, comprising administering said inducer of pyroptosis to the individual, wherein said inducer of pyroptosis is administered systemically as an adjuvant of a vaccine, preferably a genetic vaccine.

For therapeutic applications, an inducer of pyroptosis, preferably said constitutively active pro-inflammatory caspase comprising shuffled p10 and p20 domains, as is described herein is administered to an individual suffering from a disease such as a cancer or an infection in an amount sufficient to at least partially halt the disease, preferably to cure the disease, and/or to reduce or halt any disease-associated complications. An amount adequate to accomplish this is defined as a “therapeutically effective dose.” Amounts effective for this use will depend upon the severity of the disease and the general state of the individual's health and method of administration. Single or multiple administrations of an inducer of pyroptosis may be administered depending on the dosage and frequency as required and tolerated by the patient.

For prophylactic applications, an inducer of pyroptosis, preferably said constitutively active pro-inflammatory caspase comprising shuffled p10 and p20 domains, as is described herein is administered to an individual to induce an immune response that can help protect against the establishment or recurrence of a disease.

An expression construct such as a non-viral expression construct, expressing an inducer of pyroptosis, preferably said constitutively active pro-inflammatory caspase comprising shuffled p10 and p20 domains, may be formulated as an aqueous solution including a buffering agent, a saline such as sodium chloride, Ringer's dextrose, dextrose and sodium chloride, lactated Ringer's or fixed oils. Preservatives and other additives may also be present such as, for example, antimicrobials, anti-oxidants, chelating agents, and inert gases and the like.

A therapeutically effective amount of an expression construct expressing an inducer of pyroptosis, preferably said constitutively active pro-inflammatory caspase comprising shuffled p10 and p20 domains, such as a non-viral expression construct, may vary from about 0.001 mg to about 100 mg, preferably from about 0.01 mg to about 10 mg, most preferably from about 0.1 mg to about 1 mg.

Said vaccine, preferably a genetic vaccine, encodes one or more antigens, i.e. particular proteins or parts thereof, against which a protective or therapeutic immune response is desired. Said one or more antigens are either derived from a pathogen, for example a pathogen that causes a disease selected from measles, rubella, cholera, meningococcal disease, influenza, diphtheria, mumps, tetanus, hepatitis A, hepatitis B, hepatitis E, pertussis, tuberculosis, pneumococcal disease, typhoid fever, poliomyelitis, tick-borne encephalitis, Haemophilus influenzae type b, rabies, varicella and herpes zoster (shingles), human papilloma-virus, human immunodeficiency virus, respiratory syncytial virus, cytomegalovirus, rotavirus gastroenteritis, yellow fever, Japanese encephalitis, malaria, dengue fever, Zika virus-related microcephaly, anthrax, plague, Q fever, and smallpox, or are expressed by a cancer.

Examples of antigens, optionally encoded by a genetic vaccine include Human immunodeficiency virus (HIV) envelope protein (gp160), HIV-Nef, measles hemagglutinin glycoprotein, measles fusion glycoprotein, measles nucleocapsid protein, rubella E1 protein, rubella E2 protein, rubella capsid protein, cholera toxin, cholera B subunit protein, meningococcal NadA D, NHBA D, FHBP D, PorA VR1 D, PorA VR2 D and/or subvariants thereof (Brehony et al., 2015. Euro Surveill 20:10.2807/1560-7917.ES.2015.20.49.30084), influenza virus hemagglutinin, influenza virus nucleoprotein, diphtheria toxoid, mumps virus envelope glycoprotein, mumps virus hemagglutinin-neuraminidase, mumps virus hemolysis cell fusion (F) glycoprotein, mumps virus matrix envelope protein, tetanus toxoid, hepatitis A virus surface antigen, hepatitis B virus surface antigen, hepatitis E virus surface antigen, pertussis toxin, pertussis filamentous hemagglutinin, pertussis pertactin, tuberculosis ESAT-6, tuberculosis CFP10 (van Pinxteren et al., 2000. Clin Diagn Lab Immunol 7:155-160), pneumococcal capsular antigen, Salmonella Typhi O antigen, Salmonella Typhi H antigen, Salmonella Typhi 50 kDa outer membrane protein, poliovirus D antigen, poliovirus C antigen, tick-borne encephalitis virus domain III, Haemophilus influenzae type a antigen, Haemophilus influenzae type b antigen, rabies virus glycoprotein,

Varicella-Zoster virus glycoprotein E, human papilloma-virus L1 capsid protein, rotavirus E1A glycoprotein, yellow fever envelope (E) glycoprotein, Japanese encephalitis envelope protein domain III, Plasmodium falciparum glutamate dehydrogenase, histidine rich protein II, lactate dehydrogenase, and/or fructose-bisphosphate aldolase protein, dengue fever virus antigens (DEN-1 to DEN-4, Zika virus nonstructural protein 1, anthrax toxin, Yersinia pestis fraction 1 capsular antigen, Yersinia pestis fraction V protein, Q fever virus 27-kDa outer membrane protein (Com1), vaccinia virus A30, B7 and F8 antigens (Sakhatskyy et al., 2008. Virology 371:98-107), and any fragments or combinations thereof.

Preferred examples of antigens that may be encoded by a genetic vaccine are cancer neoepitopes or other tumour-associated antigens. Said cancer neoepitopes result from non-synonymous mutations, insertions or deletions in the open reading frame encoding said neoepitope in a cancer cell, resulting in altered amino acids compared to epitopes from healthy cells. Said cancer neoepitopes may differ between different cancers and individual patients. Hence, said cancer antigens preferably are developed as personalized cancer vaccines. For this, cancer cells and corresponding healthy cells are isolated from a patient, followed by sequence analysis of genomic DNA, and/or transcribed mRNAs. A comparison of the sequences obtained from cancer cells and corresponding healthy cells will result in the identification of sequences that are altered in the cancer cells, when compared to the corresponding healthy cells.

To develop said personalized cancer vaccines, software tools have been developed that detect cancer somatic mutations and predict potential tumour-specific neoepitopes. Said software tools include, but are not limited to, TSNAD: an integrated software for cancer somatic mutation and tumour-specific neoepitope detection (Zhou et al., 2017. R Soc Open Sci 4:170050); CloudNeo: a cloud pipeline for identifying patient-specific tumour neoepitopes (Bais et al., 2017. Bioinformatics, 33:3110-3112); pVAC-Seq: a genome-guided in silico approach to identifying tumour neoepitopes (Hundal et al., 2016. Genome Medicine 8:11) and pVACtools: Computational selection and visualization of neoepitopes for personalized cancer vaccine design (Kiwala et al., 2018. Cancer Genetics 226-227:45-46).

Said cancer neoepitopes can be formulated as peptides/proteins, or encoded in RNA or DNA molecules. RNA and DNA vaccines can encode several epitopes on a single molecule. Preferred genetic vaccines introduce DNA coding for cancer neoepitopes into host cells, where they are expressed and ultimately lead to the presentation of epitopes to T cells.

To stimulate a cellular immune response, antigens comprising, for example, cancer neoepitopes are preferably processed into 8- to 20-residue peptides and loaded onto a major histocompatibility complex (MHC) class I and/or class II molecules for recognition by CD8+ and/or CD4+ T cells, respectively. Said 8- to 20-amino acid residue peptides are preferably provided as a DNA expression construct encoding a preprotein, also termed polyepitope, that is processed by proteases into one or more cancer neoepitopes.

Said preprotein preferably encompasses 2-50 individual cancer neoepitopes, more preferably 2-40, more preferably 3-30, more preferably 5-25 individual cancer neoepitopes. Said individual cancer neoepitopes, or sequences comprising said neoepitopes, may be flanked by additional amino acids, and separated by small spacer sequences, preferably of 1-10 amino acid residues, preferably 1-5 amino acid residues such as 2 amino acid residues, 3 amino acid residues or 4 amino acid residues.

A genetic vaccine directs expression of said one or more antigens from an RNA or DNA expression construct. Antigen expression from a DNA expression construct is driven by a transcriptionally active promoter such as a viral promoter. Said promoter preferably is selected from a SV40 promoter, a Rous Sarcoma Virus (RSV) promoter and most preferred, a cytomegalovirus (CMV) immediate early promoter, as is known to a person skilled in the art. Additional modifications to improve expression rates include optimization of the codon usage for expression in eukaryotic cells; the insertion of promoter enhancer sequences; the insertion of synthetic introns; presence of 5′ UTR and/or 3′ UTR sequences such as adenovirus tripartite leader (TPL) sequences or Woodchuck Posttranscriptional Regulatory Element (WPRE); and modifications to the polyadenylation and transcriptional termination sequences to include a strong polyadenylation/transcriptional termination signal, such as bovine growth hormone or rabbit beta-globin polyadenylation sequences.

Said genetic vaccine may further comprise a nucleic acid encoding another immune stimulating molecule such as a cytokine. An inducer of pyroptosis, for example an active or inducible protein selected from ASC, caspase-1, and/or gasdermin D, non-limiting examples of which are provided in Tables 1-2, more preferably an expression construct as detailed herein above under 4.3, may be for use in a method of stimulating an immune response in an individual in combination with a vaccine, preferably a genetic vaccine, as described herein above, whereby said inducer of pyroptosis is administered in combination with one or more accessory molecules such as another immune stimulating molecule such as a cytokine, a chemokine, an agonistic antibody, and/or an inhibitor of immune suppressing molecules, including but not limited to an antagonistic antibody and/or a soluble ligand. Said accessory molecule may be administered as a small molecule, a protein, or as an expression construct, simultaneous, separate or sequential to the administration of the inducer of pyroptosis and the vaccine, preferably genetic vaccine.

A preferred accessory molecule is selected from an another immune stimulating molecule listed in Table 3, preferably IL-12, IL-2, IL-4, CSF2, interferon, IL-18, TNF, and/or Ox-40, most preferred IL-12 and/or CSF2, an agonistic Flt3-antibody, and/or an immune checkpoint inhibitor such as a PD1 or PD-L1 blocker such as pembrolizumab (Merck), nivolumab (Bristol-Myers Squibb), pidilizumab (Medivation/Pfizer), MEDI0680 (AMP-514; AstraZeneca) and PDR001 (Novartis); fusion proteins such as a PD-L2 Fc fusion protein (AMP-224; GlaxoSmithKline); atezolizumab (Roche/Genentech), avelumab (Merck/Serono and Pfizer), durvalumab (AstraZeneca), BMS-936559 (Bristol-Myers Squibb); and small molecule inhibitors such as PD-1/PD-L1 Inhibitor 1 (WO2015034820; (2S)-1-[[2,6-dimethoxy-4-[(2-methyl-3-phenylphenyl)methoxy]phenyl]methyl]piperidine-2-carboxylic acid), BMS202 (PD-1/PD-L1 Inhibitor 2; WO2015034820; N-[2-[[[2-methoxy-6-[(2-methyl[1,1′-biphenyl]-3-yl) methoxy]-3-pyridinyl]methyl]amino]ethyl]-acetamidc), and PD-1/PD-L1 Inhibitor 3 (WO/2014/151634; (3S,6S,12S,15S,18S,21S,24S,27S,30R,39S,42S,47aS)-3-((1H-imidazol-5-yl) methyl)-12,18-bis((1H-indol-3-yl)methyl)-N,42-bis(2-amino-2-oxoethyl)-36-benzyl-21,24-dibutyl-27-(3-guanidinopropyl)-15-(hydroxymethyl)-6-isobutyl-8,20,23,38,39-pentamethyl-1,4,7,10,13,). Further anti-PD1 molecules include ladiratuzumab vedotin (Seattle Genetics).

Said systemic administration of an inducer of pyroptosis, preferably said constitutively active pro-inflammatory caspase comprising shuffled p10 and p20 domains, as is described herein above, and the simultaneous, separate or sequential administration of a genetic vaccine as is described herein above, preferably administered as an expression construct, preferably a non-viral expression construct, preferably is parenteral such as, for example, intravenous, intraperitoneal, intranasal, intramuscular or, most preferred, intradermal.

### 4.7 Immune-Stimulating Compositions

The invention further provides an immune-stimulating composition, comprising an inducer of pyroptosis, preferably said constitutively active pro-inflammatory caspase comprising shuffled p10 and p20 domains, and a pharmacologically acceptable excipient. Said inducer of pyroptosis preferably is selected from the molecules depicted in Tables 1-2, more preferably is a constitutively active pro-inflammatory caspase comprising shuffled p10 and p20 domains.

Said pharmaceutically acceptable excipient which is a non-toxic material that does not interfere with the effectiveness of the biological activity of the active ingredient. The term “physiologically acceptable” refers to a non-toxic material that is compatible with a biological system such as a cell, cell culture, tissue, or organism. The characteristics of the carrier will depend on the route of administration. Physiologically and pharmaceutically acceptable excipients include diluents, fillers, salts buffers, stabilizers, solubilizers, and other materials which are well known in the art.

A preferred immune-stimulating composition according to the invention further comprises a genetic vaccine, preferably a genetic vaccine encoding 1-50 cancer neoepitopes as described herein above.

Said inducer of pyroptosis, preferably said constitutively active pro-inflammatory caspase comprising shuffled p10 and p20 domains, and said genetic vaccine in an immune-stimulating composition of the invention preferably are expressed from a non-viral expression construct. Non-viral expression constructs include mRNA or naked DNA such as plasmid DNA and in vitro amplified DNA. A non-viral expression construct may be packaged in liposomes and/or provided as a molecular conjugate. Minicircle DNA vectors free of plasmid bacterial DNA sequences may be generated in bacteria and may express a nucleic acid encoding an inducer of pyroptosis at high levels in vivo.

The invention further provides an immune-stimulating composition according to the invention for use in a method for treatment of a cancer.

The invention further provides a method of treating an individual suffering from a cancer, said method comprising providing an immune-stimulating composition comprising an inducer of pyroptosis, preferably said constitutively active pro-inflammatory caspase comprising shuffled p10 and p20 domains, and a genetic vaccine according to the invention to an individual in need thereof to thereby treat the individual.

The invention further provides a use of an immune-stimulating composition comprising an inducer of pyroptosis, preferably said constitutively active pro-inflammatory caspase comprising shuffled p10 and p20 domains, and a genetic vaccine according to the invention in the preparation of a medicament for treating an individual suffering from a cancer.

Said inducer of pyroptosis, preferably said constitutively active pro-inflammatory caspase comprising shuffled p10 and p20 domains, preferably is administered in combination with another immune stimulating molecule such as a cytokine such as IL-12 and CSF2.

The invention further provides a method of stimulating an immune response in an individual, preferably a T-cell mediated immune response, comprising providing an inducer of pyroptosis, preferably said constitutively active pro-inflammatory caspase comprising shuffled p10 and p20 domains, and administering said inducer of pyroptosis to the individual.

For the purpose of clarity and a concise description, features are described herein as part of the same or separate embodiments, however, it will be appreciated that the scope of the invention may include embodiments having combinations of all or some of the features described.

### 5, EXAMPLES

### Example 1

### Materials and Methods

### DNA Constructs

Expression constructs containing DNA coding for mediators of pyroptosis (Table 1) were generated by Gibson assembly. Briefly, (fragments of) Pycard, Casp1, Gsdmdc1, Il1b, I118, Ripk3, Mlkl (Mus musculus), CASP1, IL1B (Homo sapiens) were amplified from D1 (a dendritic cell line of C57BL/6 origin, see Winzler et al., 1997. J Exp Med 185:317-328) cDNA, or from synthetic, codon-optimised DNA (Pycard, Csf2, hCASP1_RV2; Integrated DNA Technologies, Coralville, IA, USA) and cloned into vector pD2610-v10 (ATUM, Newark, CA, USA) using a Gibson Assembly Cloning Kit (New England Biolabs, Ipswich, MA, USA). Constructs encoding dasher GFP (SEQ ID NO: 13), Reps1, a small, irrelevant peptide sequence (SEQ ID NO: 20), or lacking an insert altogether, served as controls.

In vaccination experiments, adjuvant or control plasmids were combined with a polyepitope DNA vaccine (SEQ ID NO: 23) coding for three tumour-specific antigens (Dpagt1, Reps1, Adpgk; see SEQ ID NO: NOs: 25-27) derived from the C57BL/6 MC38 colon carcinoma cell line, as well as two model antigens (OT-II, OT-I; see SEQ ID NO: NOs: 28 and 29) from chicken ovalbumin, separated by a ‘spacer sequence’ consisting of three alanines (see SEQ ID NO: 30).

All plasmids were grown using E. coli strain DH5α and purified using a Macherey-Nagel (Dueren, Germany) Endotoxin-Free (EF) plasmid purification kit. For vaccination, plasmids underwent an additional purification step on a Nucleobond filter, followed by centrifugation (30 min, 10,000 g, 4° C.) to remove any remaining debris.

### Mice and Cell Lines

C57BL/6 (Jico) mice were purchased from Jackson laboratory (Bar Harbor, ME, USA) and housed under FELASA-compliant conditions at the LUMC animal facility. B16-F10, a melanoma cell line of C57BL/6 origin, was maintained in culture medium consisting of IMDM (ThermoFisher-Gibco, Waltham, MA, USA) supplemented with 8% fetal calf serum (Sigma-Aldrich, Zwijndrecht, the Netherlands) in the presence of L-glutamine, penicillin and streptomycin (all from ThermoFisher-Gibco) in a humidified CO2-incubator (37° C., 5% CO2). B16-OVA is a B16-F10 cell line stably transfected with ovalbumin and was cultured under the same conditions.

### Transfections

B16-F10 cells were plated at 2,000 cells/well in a 96-well flat bottom plate in 100 μl culture medium. One day later, they were transfected by the addition of 10 μl DNA complexed with Saint-DNA (Synvolux products, Leiden, the Netherlands), a cationic lipid-based transfection reagent. Two days after transfection, cell death and/or the release of DAMPS were analysed by flow cytometry, ELISA or LDH assay.

### Flow Cytometry

Non-adherent and adherent cells were harvested by removing the cell supernatants, rinsing the wells with PBS and subsequently treating the remaining adherent cells with trypsin (ThermoFisher-Gibco). After mixing and centrifugation of the supernatant and trypsin-treated cells in 96-well V-bottom plates, the resulting cells were washed with FACS buffer (PBS, 1% BSA, 0.02% azide) and exposed to 7-aminoactinomycin D (7-AAD) (Biolegend, San Diego, CA, USA), a dye that enters dead cells that have lost membrane integrity, diluted in FACS buffer for 15 minutes. The cells were then immediately analysed on a Guava EasyCyte HT flow cytometer equipped with a 488 nm laser (Merck MilliPore, Burlington, MA, USA)

### IL-1β ELISA

To measure IL-1β, cellular supernatants were centrifuged to remove cell debris and analysed by sandwich ELISA, using protocols provided by the supplier (Biolegend, San Diego, CA). Briefly, 96-well plates were coated with capture antibody overnight. After washing away unbound antibody (4 washing steps), 50-1000-fold diluted supernatant (and, for reference, titrated recombinant IL-1β) was added and incubated for 2 h at room temperature while shaking. After 4 washing steps, biotinylated detection antibody was added and incubated for 1 h at RT, followed by another 4 wash steps and a 30-minute incubation with streptavidin-HRP. After washing away strep-HRP, TMB substrate solution was added. Absorbance at 450 nm and 570 nm was read on a Tecan Infinite F50 (Tecan Group Ltd, Männedorf, Switzerland).

### LDH Assay

A colorimetric assay to quantitatively measure lactate dehydrogenase (LDH) released into the culture media (LDH Cytotoxicity Assay Kit, ThermoFisher-Pierce, Waltham, MA, USA) was used to quantify cell death. This assay is based on a coupled enzymatic reaction. First, LDH catalyzes the conversion of lactate to pyruvate via reduction of NAD+ to NADH. Second, diaphorase uses NADH to reduce a tetrazolium salt (INT) to a red formazan product. Therefore, the level of formazan formation is directly proportional to the amount of released LDH in the medium. Briefly, 50 ul reaction mixture was mixed with 50 ul 2-fold diluted culture supernatant. After 30 minutes incubation at room temperature in the dark, stop solution was added, followed by absorbance readings at 450 nm and 620 nm. Percentage cytotoxicity was calculated as follows: ((OD450−OD620)sample−(OD450−OD620)medium control)/((OD450−OD620)positive control−(OD450−OD620)medium control)×100%.

### Mouse Vaccinations and Tumour Challenge

On day 0, male C57BL/6 mice were injected intradermally with 30 μl 0.9% NaCl solution containing 30 μg endotoxin-free plasmid DNA, consisting of 10 μg polyepitope vaccine (see SEQ ID NO: NO: 23) (or empty vector control), 10 μg adjuvant (or empty vector), and 10 μg adjuvant 2 (or empty vector). Blood, drawn on several days after vaccination, was treated with erythrocyte lysis buffer and stained with PE-conjugated H2-Kb/SIINFEKL (SEQ ID NO: 65) tetramers (LUMC tetramer facility, Leiden, the Netherlands) in PBS supplemented with 0.1% bovine serum albumin and 0.02% sodium azide (PBS/BSA). After a 30-minute incubation at room temperature in the dark, fluorochrome-conjugated antibodies to CD3, CD4, CD8 (Biolegend, San Diego, CA, USA) were added to discriminate T cell subsets, followed by another 30 minutes on ice and 2 washing steps with PBS/BSA to remove unbound tetramers and antibodies. The samples were acquired on a BD LSRII (Becton Dickinson, San Jose, CA, USA) and analysed using FlowJo (FlowJo LLC).

On day 29 after vaccination, mice were injected subcutaneously with 50,000 B16-OVA cells, B16-F10 melanoma cells stably transfected with ovalbumin. Tumour growth was monitored every 3-4 days, and tumour size was calculated as (length×width×width)/2. Mice carrying tumours exceeding 1000 mm3 or with a bleeding ulcer were euthanised by CO2 asphyxiation.

### Results

### Design of Constitutively Active Caspase-1 Variants

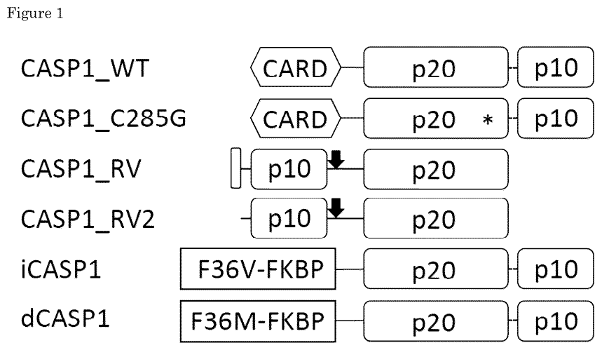

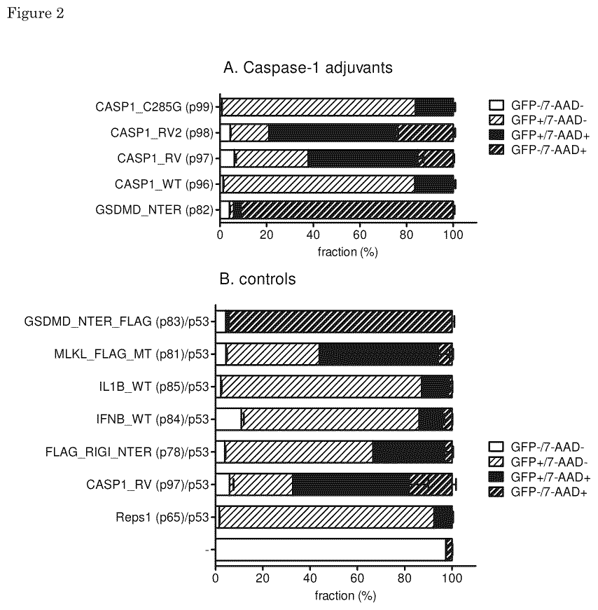

A series of constructs based on ASC (SEQ ID NO: 31), caspase-1 (SEQ ID NO: 1-4, 8, 12), gasdermin D (SEQ ID NO: 14-15), signature signaling molecules in pyroptosis, cDNA sequences was designed (FIG. 1, Table 1, 2, 4). Work using non-inflammatory caspases (Srinivasula et al., 1998. J Biol Chem 273:10107-11; Park et al., 2006. Biochem Biophys Res Commun 347:941-8) suggested that a protein consisting of the caspase-1 p10 and p20 domains, in reverse order compared to wild-type Caspase-1 (‘reshuffled’) and linked by the IL-1β caspase-1 cleavage site would be constitutively active. However, a recent study shows that the active form of caspase-1 also requires the presence of the N-terminal CARD domain (Boucher et al., 2018. J Exp Med 215:827-840), suggesting that such constructs (CASP1_RV and CASP1_RV2, FIG. 1; SEQ ID NO: 3 and 4, respectively) would not be active. In additional constructs, inducible (iCASP1, SEQ ID NO: 8) or constitutive (dCASP1, SEQ ID NO: 12), dimerisation domains replaced the N-terminal CARD domain that normally links caspase-1 to upstream activating signaling cascades. Finally, a control construct with a mutated active site (CASP1_C285G, SEQ ID NO: 2) as well as a wild-type control (CASP1_WT, SEQ ID NO: 1) was generated.

### Constitutively Active Caspase-1 Variants CASP1_RV and CASP1_RV2 Induce Cell Death

Upon activation, caspase-1 induces pyroptotic cell death by cleaving gasdermin D (Aglietti and Dueber, 2017. Trends Immunol 38:261-271). This releases the gasdermin D N-terminal domain, causing it to form cytotoxic pores in the plasma membrane of cells. To test if our caspase-1 variants (FIG. 1) induce cell death, B16F10 cells were transfected with the corresponding plasmids together with a GFP-encoding plasmid (SEQ ID NO: 13). Two days after transfection, wild-type caspase-1 (CASP1_WT) and the active site mutant (CASP1_C285G) had induced little to no cell death (<20% 7-AAD+ cells) compared to the negative control (SEQ ID NO: 20), while the N-terminal domain of GSDMD (SEQ ID NO: 14-15) killed virtually all (>90%) cells (FIG. 2). In contrast with wild-type and point-mutated caspase-1, the ‘reshuffled’ caspase-1 (CASP1_RV and CASP1_RV2) variants killed the large majority of the B16F10 cells. In addition, most of the dead cells expressed GFP, suggesting that inclusion of active caspase-1 in a genetic vaccine does not prevent the expression of antigen encoded by a DNA vaccine. Thus, in contrast with an earlier report indicating that active caspase-1 requires the CARD domain (Boucher et al., 2018. J Exp Med 215:827-840), the novel CARD-less ‘reshuffled’ caspase-1 constructs of the present invention were shown to be constitutively active.

### Constitutively Active Caspase-1 Variants CASP1_RV and CASP1_RV2 Induce IL-1β Secretion

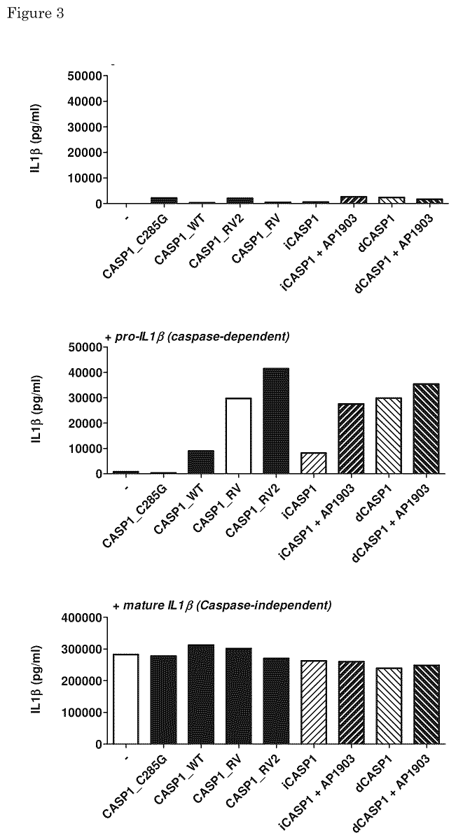

One of the hallmarks of pyroptosis is the secretion of IL-1β, a pyrogenic cytokine. This cytokine is produced as a cytosolic precursor (pro-IL-1β) and its release requires cleavage by caspase-1 of both pro-IL-1β and gasdermin D (Evavold et al., 2018. Immunity 48:35-44; Heilig et al., 2018. Eur J Immunol 48:584-592; Monteleone et al., 2018. Cell Rep 24:1425-1433). Indeed, the active caspase-1 variants CASP1_RV and CASP1_RV2, but not CASP1_WT and CASP1_C285G, induced IL-1β release from B16-F10 cells co-transfected with pro-IL-1β (SEQ ID NO: 21, FIG. 3). Similarly, caspase-1 dimers, either induced by AP1903 (iCASP1) or constitutive (dCASP1), also yielded IL-1β secretion in this assay.

### CASP1_RV2 Improves T Cell Responses and Anti-Tumour Immunity

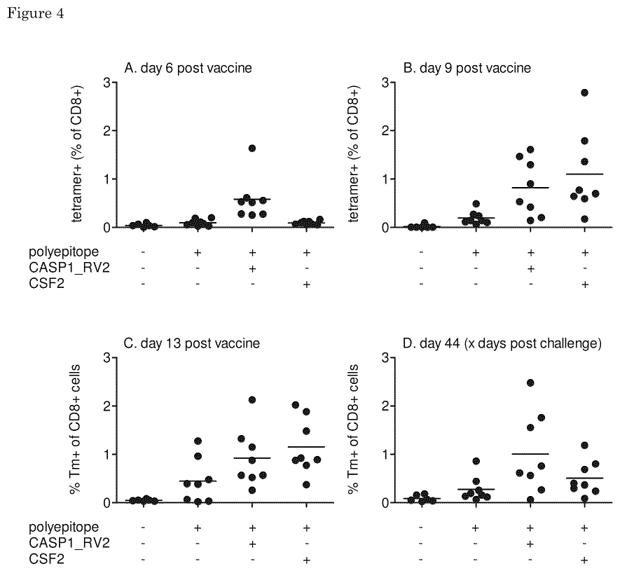

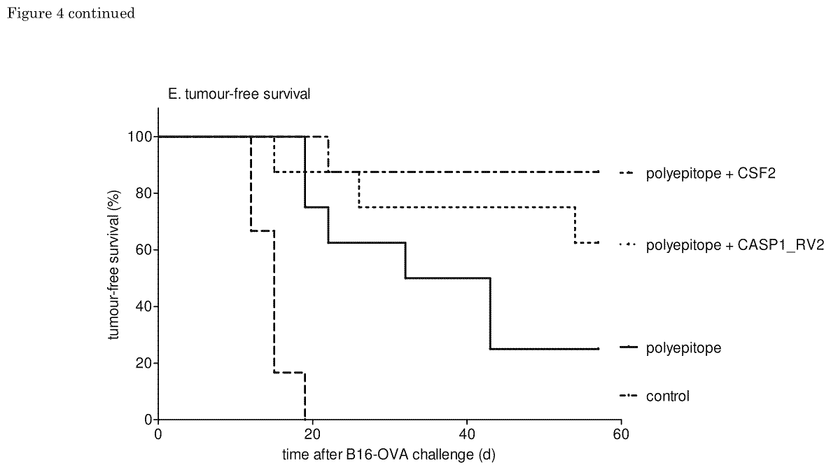

As the constitutively active forms of caspase-1 (CASP1_RV and CASP1_RV2) induced cell death and were able to induce processing and secretion of IL-1β, we next tested their potential as a genetic adjuvant. To this end, mice were vaccinated with a 1:1 mix of a plasmid coding for a polyepitope vaccine (SEQ ID NO: 23) and a plasmid encoding a genetic adjuvant. The polyepitope vaccine included the ovalbumin-derived CD8+ T cell epitope SIINFEKL (SEQ ID NO: 65), that is recognised by specific T cells when bound to H-2Kb MHC class I molecule. Since CASP1_RV2 was slightly more active than CASP1_RV (FIG. 2-3), the former was used. Shortly after vaccination, CASP1_RV2, but not reference adjuvant CSF2, significantly increased the frequency of SIINFEKL (SEQ ID NO: 65)-specific T cells (FIG. 4A) found in blood, while at later time points the adjuvant effects of CASP1_RV2 and CSF2 were similar (FIG. 4B, C).

Intriguingly, challenging the mice with B16-OVA resulted in a massive increase in specific T cell immunity in the CASP1_RV2-adjuvanted group compared to mice that had not received an adjuvant, much greater than for CSF2 (SEQ ID NO: 24, FIG. 4D). This may suggest that this adjuvant works particularly well in heterologous prime-boost regimens (Kardani et al., 2016, Vaccine 34:413-423).

In mock-vaccinated mice, B16-OVA tumours invariably grew out within three weeks after injection (FIG. 4E). Although the polyepitope vaccine significantly delayed tumour growth, most of the mice eventually did develop a tumour. In contrast, the majority of CASP1_RV2 adjuvanted mice remained tumour-free. Thus, a new constitutively active form of caspase-1 significantly improved both T-cell immunity and tumour protection.

### Constitutively Active Forms of RIG-I and GSDMD Do Not Improve T Cell Immunity

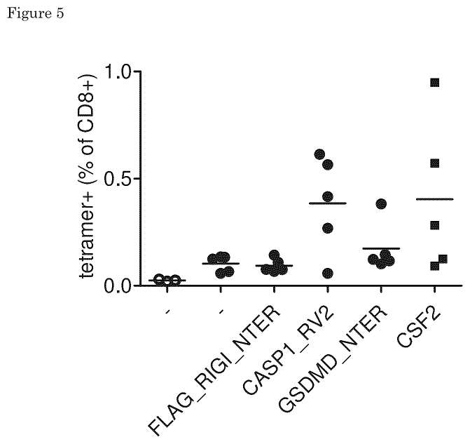

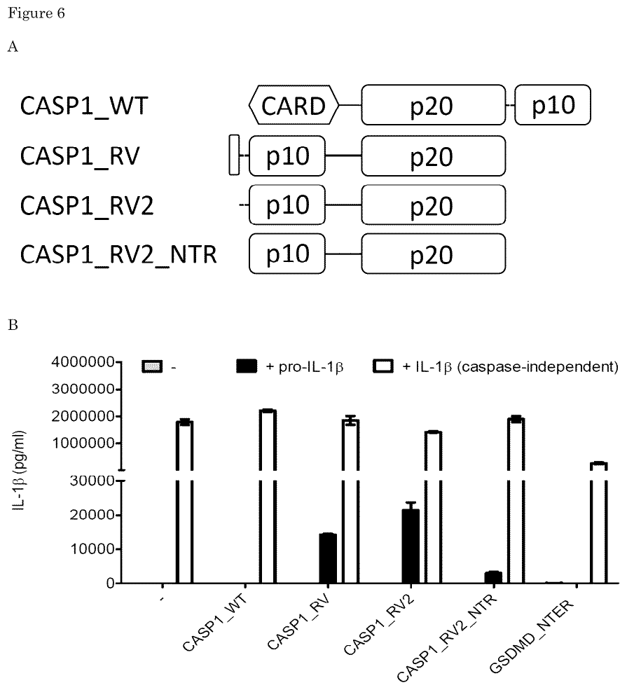

Caspase-1 is a central part of the inflammasome pathway. Therefore, we reasoned that other components of this pathway should be also be able to act as genetic adjuvants. To test this idea, we generated a constitutively active version of Ddx58, also known as RIG-I (SEQ ID NO: 19), and of Gsdmdc1, also known as GSDMD (SEQ ID NO: 14), by removing their C-terminal inhibitory domains. RIG-I can act upstream of caspase-1 to promote IL-1β processing and release (Poeck et al. 2010, Nature Immunology 11:63-69), and GSDMD is a downstream target of caspase-1 responsible for membrane disruption. These constructs both induced cell death (FIG. 2). In addition, and in contrast with all other constructs tested, transfection of B16F10 cells with the RIG-I construct resulted in production of IFNβ and IL-6 (data not shown). Surprisingly, however, when tested for their adjuvant activity in combination with the polyepitope vaccine, only the active caspase-1 construct improved T cell responses (FIG. 5).

### Example 2

### Materials and Methods

### DNA Constructs

Expression constructs containing DNA coding for mediators of pyroptosis (Table 1 and Table 4) were generated by Gibson assembly. Briefly, (fragments of) Casp1, Gsdmdc1, Il1b, (Mus musculus), IL1B, GSDMD (Homo sapiens) were amplified from D1 (a dendritic cell line of C57BL/6 origin, see Winzler et al., 1997. J Exp Med 185:317-328) or 293 cDNA, or from synthetic, codon-optimised DNA (CASP1; Integrated DNA Technologies, Coralville, IA, USA) and cloned into vector pD2610-v10 (ATUM, Newark, CA, USA) using a Gibson Assembly Cloning Kit (New England Biolabs, Ipswich, MA, USA). Small alterations in these constructs (e.g. modifications at the N-terminus or linker sequence, single amino acid substitutions) were introduced using dedicated primers and Gibson assembly. All plasmids were grown using E. coli strain DH5α and purified using a Macherey-Nagel (Dueren, Germany) Endotoxin-Free (EF) plasmid purification kit.

### Cell Lines

B16-F10, a melanoma cell line of C57BL/6 origin, was maintained in culture medium consisting of IMDM (ThermoFisher-Gibco, Waltham, MA, USA) supplemented with 8% fetal calf serum (Sigma-Aldrich, Zwijndrecht, the Netherlands) in the presence of L-glutamine, penicillin and streptomycin (all from ThermoFisher-Gibco) in a humidified CO2-incubator (37 ° C., 5% CO2). 293, an epithelial cell line derived from human embryonic kidney cell (ATCC CRL-1573), was cultured under the same conditions.

### Transfections

B16-F10 cells (2,000 cells/well) or 293 cells (20,000 cells/well) were plated in a 96-well flat bottom plate in 100 μl culture medium. One day later, they were transfected by the addition of 10 μl (20-40 ng) DNA complexed with Saint-DNA (Synvolux products, Leiden, the Netherlands), a cationic lipid-based transfection reagent. Two days after transfection, cell death and/or the release of DAMPS were analysed by LDH assays or ELISA.

### IL-1β ELISA

To measure IL-1β, cellular supernatants were centrifuged to remove cell debris and analysed by sandwich ELISA, using protocols provided by the supplier (Biolegend, San Diego, CA). Briefly, 96-well plates were coated with capture antibody overnight. After washing away unbound antibody (4 washing steps), 50-1000-fold diluted supernatant (and, for reference, titrated recombinant IL-1β) was added and incubated for 2 h at room temperature while shaking. After 4 washing steps, biotinylated detection antibody was added and incubated for 1 h at RT, followed by another 4 wash steps and a 30-minute incubation with streptavidin-HRP. After washing away strep-HRP, TMB substrate solution was added. Absorbance at 450 nm and 570 nm was read on a Tecan Infinite F50 (Tecan Group Ltd, Männedorf, Switzerland).

### LDH Assay

The LDH-Glo Cytotoxicity assay (Promega, Madison, WI, USA) was used to measure LDH activity in culture supernatants. In this assay, LDH released from damaged cells catalyzes the oxidation of lactate with concomitant reduction of NAD+ to NADH. Reductase uses NADH and reductase substrate to generate luciferin, which is converted to a bioluminescent signal by Ultra-Glo™ rLuciferase. The luminescent signal generated is proportional to the amount of LDH present. Briefly, 10 ul culture supernatant was diluted with 90 ul LDH storage buffer (200 mM Tris-HCl (pH 7.3), 10% Glycerol, 1% BSA). After another 10-fold dilution in LDH storage buffer, 50 ul 100-fold diluted supernatant was mixed with 50 ul LDH Detection Reagent and incubated at room temperature in the dark, followed by luminescence readings at 30 and 60 minutes on a Tecan Infinite F50 (Tecan Group Ltd, Männedorf, Switzerland). Data were represented as Relative Light Units (RLU).

### Results

### Murine CASP1_RV2 Functionality Depends on Presence N-Terminal IDL Sequence

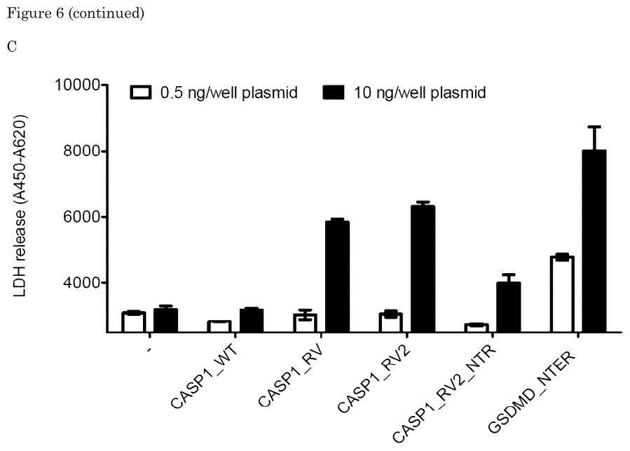

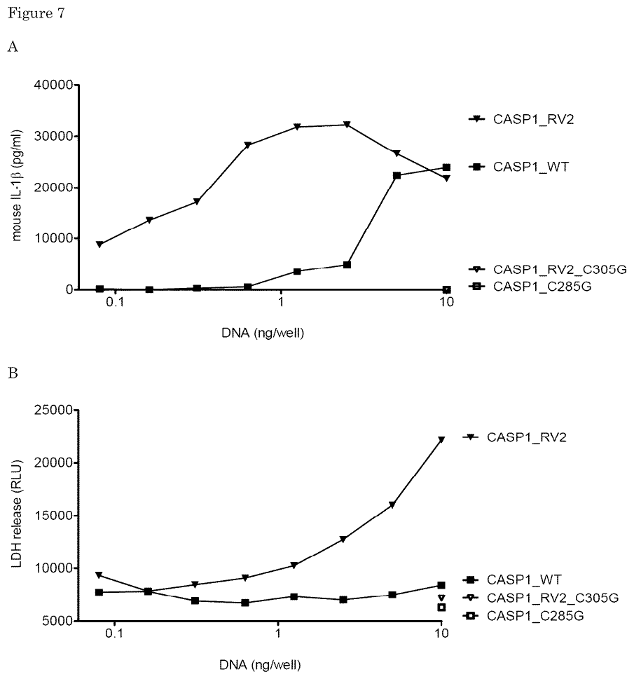

Our initial design of the constitutively active murine inflammatory caspase-1 (CASP1_RV, SEQ ID NO: 3) was led by the design of constitutively active versions of human apoptosis-inducing human executioner caspase-3 and-6 (Srinivasula et al., 1998. J Biol Chem 273:10107-11). In the design of active caspase-3, for example, the N-terminal p20 and C-terminal p10 domains were swapped and separated by a short (8 AA) caspase-3 cleavage site. A part of the sequence upstream of p10, including several p20 amino acids, was also moved by this swap, the N-terminus of resulting active caspase began with four p20 amino acids, followed by the small (6 AA) p20-p10 interdomain linker and p10 (Srinivasula et al., 1998. J Biol Chem 273:10107-11). Accordingly, the N-terminus of murine CASP1_RV began with five p20 amino acids and the IDL. As removal of the p20 remnant in CASP1_RV2 (SEQ ID NO: 4) appeared to increase activity compared to CASP1_RV (SEQ ID: 3, FIGS. 2-3), we tested an additional variant CASP1_RV2_NTR (SEQ ID NO: 38) lacking the IDL altogether. Testing these three CASP1_RV variants progressively lacking more of this N-terminus showed that removal of the N-terminal p20 remnant in CASP1_RV2 increased, but further removal of the IDL in CASP1_RV2_NTR decreased the activity of CASP1_RV (FIG. 6). In other words, the most active CASP1_RV2 variant retained most of the, highly negatively charged, IDL sequence SEEDFLTDAIFEDD (SEQ ID NO: 60) at its N-terminus. Compared to wild-type murine caspase-1 (CASP1_WT), CASP1_RV2 was approximately 30-fold more potent in in vitro assays (FIG. 7). Thus, despite crucial differences between human caspase-3 and mouse caspase-1, such as the presence of a CARD domain in caspase-1 and a difference in activation mechanism, the Srinivasula-approach indeed generated constitutively active mouse caspase-1.

### Human CASP1_RV2 Functionality Relies on Absence N-Terminal IDL Sequence

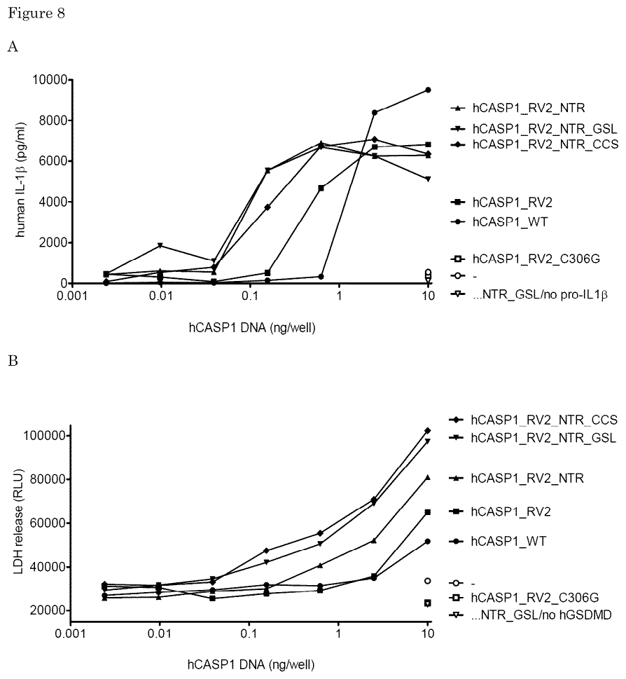

The design of a constitutively active human caspase-1 variant (hCASP1_RV2, SEQ ID NO: 35) was based on murine CASP1_RV2, and therefore also began with 14 amino acids of the interdomain linker (IDL): GNLSLPTTEEFEDD (SEQ ID NO: 61). However, hCASP1_RV2 was not significantly more active than wild-type human caspase-1 (hCASP1_WT, FIG. 8). Thus, in contrast with mouse caspase-1, following the Srinivasula-approach did not significantly increase the activity of human caspase-1. Surprisingly, in striking contrast with murine CASP1_RV2 (FIG. 6) and despite its distance from the enzymatically active site (Yang et al., 2018. Proc Natl Acad Sci USA 115:6792-6797), removing all N-terminal residues upstream of p10 (hCASP1_RV2_NTR, SEQ ID NO: 49) did greatly increase its activity (FIG. 8).

Reduction of p10-p20 Linker Size Further Increases Constitutively Active Human Caspase-1 Potency

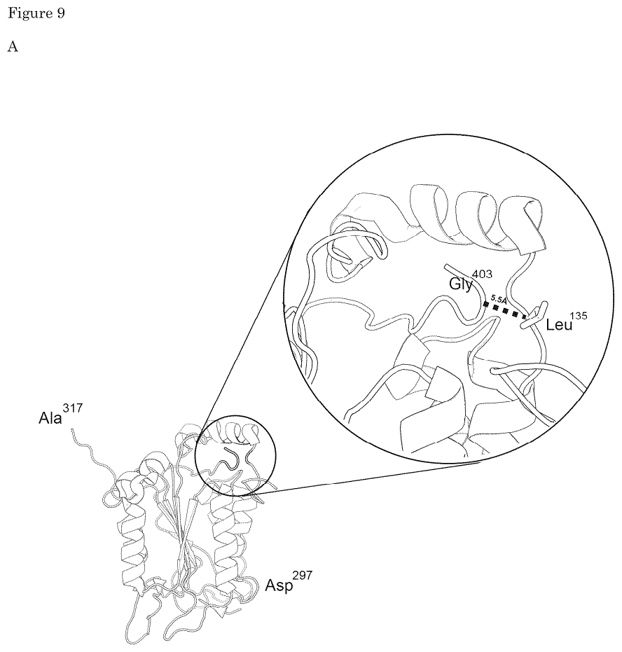

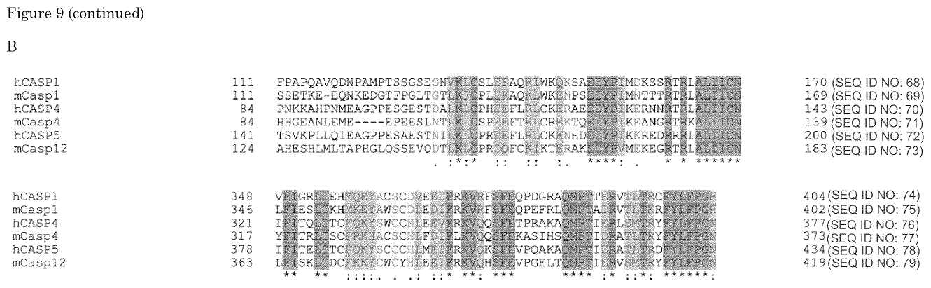

Next, we turned our attention to the intervening sequence between p10 and p20. Removing aspartic acid protease sites in this region of mouse and human CASP1_RV2 [SEQ ID NOs 40-43, 45-47] did not significantly affect their ability to process pro-IL1β, suggesting that autoproteolysis did not contribute to its activity (data not shown). Rather, this region appeared to serve merely as a flexible linker. Crystal structures of caspase-1 in its active conformation (Yang et al., 2018. Proc Natl Acad Sci USA 115:6792-6797) indicated that the distance between the C-terminus of the p10 domain and the N-terminus of the p20 domain is rather small (FIG. 9). In fact, we discovered that reducing the size of the linker to 9 amino acids was not a neutral event, as we had expected, but surprisingly increased the activity of hCASP1_NTR (FIG. 8B). This was independent of the presence (AYVHDAPVR (SEQ ID NO: 66) in hCASP1_NTR_CCS, SEQ ID NO: 51) or absence (GSGSGSGSG (SEQ ID NO: 67) in hCASP1_NTR_GSL, SEQ ID NO: 50) of an autoproteolytic target site in this smaller linker (FIG. 8B). Compared to wild-type human caspase-1, hCASP1_NTR_GSL (SEQ ID NO:50) was approximately 30-fold more potent in in vitro assays (FIG. 8). Thus, while the Srinivasula-approach did not significantly increase the activity of human caspase-1, removing N-terminal residues and, to a lesser extent, reducing linker size unexpectedly did yield a constitutively active human caspase-1.

TABLE 1
Pyroptosis-associated constructs. For a detailed description
of caspase-1 constructs, see FIG. 1.

| | | | |
|---|---|---|---|
| | | | SEQ |
| Gene | | | ID |
| name | Construct | Modification | NO |

Mus musculus

| | | | |
|---|---|---|---|
| Pycard | dCARD | ASC CARD domain + 2 | 31 |
| | | C-terminal dimerisation | |
| | | domains | |
| Casp 1 | CASP1_C285G | active site mutant | 2 |
| | CASP1_WT | wild-type (wt) | 1 |
| | CASP1_RV | p10 and p20 domain | 3 |
| | | reversal | |
| | CASP1_RV2 | p10 and p20 domain | 4 |
| | | reversal | |
| | dCASP1 | wt + 2 N-terminal | 12 |
| | | dimerisation domains | |
| | iCASP1 | wt + 2 N-terminal AP1903- | 8 |
| | | inducible dimerisation | |
| | | domains | |
| Gsdmdc1 | GSDMD_WT_FLAG | wild-type (+FLAG) | 32 |
| | GSDMD_NTER | N-terminal fragment | 14 |
| | GSDMD_NTER_FLAG | N-terminal fragment | 15 |
| | | (+FLAG) | |
| Il1b | IL1B_WT | mature IL-1β (IFNβ signal | 17 |
| | | peptide) | |
| | IL1B_FL | wild-type pro-IL1β | 21 |
| | IL1B_CALRSP | mature IL-1β (Calreticulin | 33 |
| | | signal peptide) | |
| Il18 | IL18_WT | mature IL-18 (IFNβ signal | 34 |
| | | peptide) | |

Homo sapiens

| | | | |
|---|---|---|---|
| CASP1 | hCASP1_RV2 | human version | 35 |
| | | CASP1_RV2 | |
| IL1B | hIL1B_SP | human mature IL-1β (IFNβ | 36 |
| | | SP) | |
| | hIL1B_FL | human wild-type pro-IL1β | 37 |

TABLE 2
Amino acid sequences.

| SEQ ID | | | |
|---|---|---|---|
| NO | Gene | Construct | AA sequence |
| 1 | Casp1 | CASP1_WT | MADKILRAKRKQFINSVSIGTINGLLDELLEKRVLNQEE |
| | | | MDKIKLANITAMDKARDLCDHVSKKGPQASQIFITYICN |
| | | | EDCYLAGILELQSAPSAETFVATEDSKGGHPSSSETKEE |
| | | | QNKEDGTFPGLTGTLKFCPLEKAQKLWKENPSEIYPIMN |
| | | | TTTRTRLALIICNTEFQHLSPRVGAQVDLREMKLLLEDL |
| | | | GYTVKVKENLTALEMVKEVKEFAACPEHKTSDSTFLVFM |
| | | | SHGIQEGICGTTYSNEVSDILKVDTIFQMMNTLKCPSLK |
| | | | DKPKVIIIQACRGEKQGVVLLKDSVRDSEEDFLTDAIFE |
| | | | DDGIKKAHIEKDFIAFCSSTPDNVSWRHPVRGSLFIESL |
| | | | IKHMKEYAWSCDLEDIFRKVRFSFEQPEFRLQMPTADRV |
| | | | TLTKRFYLFPGH |
| 2 | Casp1 | CASP1_C285G | MADKILRAKRKQFINSVSIGTINGLLDELLEKRVLNQEE |
| | | | MDKIKLANITAMDKARDLCDHVSKKGPQASQIFITYICN |
| | | | EDCYLAGILELQSAPSAETFVATEDSKGGHPSSSETKEE |
| | | | QNKEDGTFPGLTGTLKFCPLEKAQKLWKENPSEIYPIMN |
| | | | TTTRTRLALIICNTEFQHLSPRVGAQVDLREMKLLLEDL |
| | | | GYTVKVKENLTALEMVKEVKEFAACPEHKTSDSTFLVFM |
| | | | SHGIQEGICGTTYSNEVSDILKVDTIFQMMNTLKCPSLK |
| | | | DKPKVIIIQAGRGEKQGVVLLKDSVRDSEEDFLTDAIFE |
| | | | DDGIKKAHIEKDFIAFCSSTPDNVSWRHPVRGSLFIESL |
| | | | IKHMKEYAWSCDLEDIFRKVRFSFEQPEFRLQMPTADRV |
| | | | TLTKRFYLFPGH |
| 3 | Casp1 | CASP1_RV | MVLLKDSVRDSEEDFLTDAIFEDDGIKKAHIEKDFIAFC |
| | | | SSTPDNVSWRHPVRGSLFIESLIKHMKEYAWSCDLEDIF |
| | | | RKVRFSFEQPEFRLQMPTADRVTLTKRFYLFPGHLLVCD |
| | | | VPIRAPSAETFVATEDSKGGHPSSSETKEEQNKEDGTFP |
| | | | GLTGTLKFCPLEKAQKLWKENPSEIYPIMNTTTRTRLAL |
| | | | IICNTEFQHLSPRVGAQVDLREMKLLLEDLGYTVKVKEN |
| | | | LTALEMVKEVKEFAACPEHKTSDSTFLVFMSHGIQEGIC |
| | | | GTTYSNEVSDILKVDTIFQMMNTLKCPSLKDKPKVIIIQ |
| | | | ACRGEKQGVVLLKD |
| 4 | Casp1 | CASP1_RV2 | MSEEDFLTDAIFEDDGIKKAHIEKDFIAFCSSTPDNVSW |
| | | | RHPVRGSLFIESLIKHMKEYAWSCDLEDIFRKVRFSFEQ |
| | | | PEFRLQMPTADRVTLTKRFYLFPGHLLVCDVPIRAPSAE |
| | | | TFVATEDSKGGHPSSSETKEEQNKEDGTFPGLTGTLKFC |
| | | | PLEKAQKLWKENPSEIYPIMNTTTRTRLALIICNTEFQH |
| | | | LSPRVGAQVDLREMKLLLEDLGYTVKVKENLTALEMVKE |
| | | | VKEFAACPEHKTSDSTFLVFMSHGIQEGICGTTYSNEVS |
| | | | DILKVDTIFQMMNTLKCPSLKDKPKVIIIQACRGEKQGV |
| | | | VLLKD |
| 5 | Casp1 | S, p10 | GIKKAH IEKDFIAFCS STPDNVSWRH PVRGSLFIES |
| | | (AA315- | LIKHMKEYAW SCDLEDIFRK VRFSFEQPEF |
| | | 402) | RLQMPTADRV TLTKRFYLFP GH |
| 6 | Casp1 | CARD-L | APSAETFVA TEDSKGGHPS SSETKEEQ |
| | | linker | |
| | | (AA92- | |
| | | 118) | |
| 7 | Il1b | Illb | LLVCDVPIR |
| | | caspase-1 | |
| | | cleavage | |
| | | site | |
| 8 | Casp1 | iCASP1 | MGSRGVQVETISPGDGRTFPKRGQTCVVHYTGMLEDGKK |
| | | | VDSSRDRNKPFKFMLGKQEVIRGWEEGVAQMSVGQRAKL |
| | | | TISPDYAYGATGHPGIIPPHATLVFDVELLKLETRGVQV |
| | | | ETISPGDGRTFPKRGQTCVVHYTGMLEDGKKVDSSRDRN |
| | | | KPFKFMLGKQEVIRGWEEGVAQMSVGQRAKLTISPDYAY |
| | | | GATGHPGIIPPHATLVFDVELLKLESGGGSAPSAETFVA |
| | | | TEDSKGGHPSSSETKEEQNKEDGTFPGLTGTLKFCPLEK |
| | | | AQKLWKENPSEIYPIMNTTTRTRLALIICNTEFQHLSPR |
| | | | VGAQVDLREMKLLLEDLGYTVKVKENLTALEMVKEVKEF |
| | | | AACPEHKTSDSTFLVFMSHGIQEGICGTTYSNEVSDILK |
| | | | VDTIFQMMNTLKCPSLKDKPKVIIIQACRGEKQGVVLLK |
| | | | DSVRDSEEDFLTDAIFEDDGIKKAHIEKDFIAFCSSTPD |
| | | | NVSWRHPVRGSLFIESLIKHMKEYAWSCDLEDIFRKVRF |
| | | | SFEQPEFRLQMPTADRVTLTKRFYLFPGH |
| 9 | n.a. | SGGGS | SGGGS |
| | | linker | |
| 10 | n.a. | F36V-FKBP | MGSRGVQVET ISPGDGRTFP KRGQTCVVHY |
| | | | TGMLEDGKKV DSSRDRNKPF KFMLGKQEVI |
| | | | RGWEEGVAQM SVGQRAKLTI SPDYAYGATG |
| | | | HPGIIPPHAT LVFDVELLKL ETRGVQVETI |
| | | | SPGDGRTFPK RGQTCVVHYT GMLEDGKKVD |
| | | | SSRDRNKPFK FMLGKQEVIR GWEEGVAQMS |
| | | | VGQRAKLTIS PDYAYGATGH PGIIPPHATL |
| | | | VFDVELLKLE |
| 11 | n.a. | F36M-FKBP | MGVQVET ISPGDGRTFP KRGQTCVVHY |
| | | | TGMLEDGKKM DSSRDRNKPF KFMLGKQEVI |
| | | | RGWEEGVAQM SVGQRAKLTI SPDYAYGATG |
| | | | HPGIIPPHAT LVFDVELLKL ETRGVQVETI |
| | | | SPGDGRTFPK RGQTCVVHYT GMLEDGKKMD |
| | | | SSRDRNKPFK FMLGKQEVIR GWEEGVAQMS |
| | | | VGQRAKLTIS PDYAYGATGH PGIIPPHATL |
| | | | VFDVELLKLE |
| 12 | Casp1 | dCASP1 | MGVQVETISPGDGRTFPKRGQTCVVHYTGMLEDGKKMDS |
| | | | SRDRNKPFKFMLGKQEVIRGWEEGVAQMSVGQRAKLTIS |
| | | | PDYAYGATGHPGIIPPHATLVFDVELLKLETRGVQVETI |
| | | | SPGDGRTFPKRGQTCVVHYTGMLEDGKKMDSSRDRNKPF |
| | | | KFMLGKQEVIRGWEEGVAQMSVGQRAKLTISPDYAYGAT |
| | | | GHPGIIPPHATLVFDVELLKLESGGGSAPSAETFVATED |
| | | | SKGGHPSSSETKEEQNKEDGTFPGLTGTLKFCPLEKAQK |
| | | | LWKENPSETYPIMNTTTRTRLALIICNTEFQHLSPRVGA |
| | | | QVDLREMKLLLEDLGYTVKVKENLTALEMVKEVKEFAAC |
| | | | PEHKTSDSTFLVFMSHGIQEGICGTTYSNEVSDILKVDT |
| | | | IFQMMNTLKCPSLKDKPKVIIIQACRGEKQGVVLLKDSV |
| | | | RDSEEDFLTDAIFEDDGIKKAHIEKDFIAFCSSTPDNVS |
| | | | WRHPVRGSLFIESLIKHMKEYAWSCDLEDIFRKVRFSFE |
| | | | QPEFRLQMPTADRVTLTKRFYLFPGH |
| 13 | n.a. | dasher | MTALTEGAKLFEKEIPYITELEGDVEGMKFIIKGEGTGD |
| | | GFP | ATTGTIKAKYICTTGDLPVPWATLVSTLSYGVQCFAKYP |
| | | | SHIKDFFKSAMPEGYTQERTISFEGDGVYKTRAMVTYER |
| | | | GSIYNRVTLTGENFKKDGHILRKNVAFQCPPSILYILPD |
| | | | TVNNGIRVEFNQAYDIEGVTEKLVTKCSQMNRPLAGSAA |
| | | | VHIPRYHHITYHTKLSKDRDERRDHMCLVEVVKAVDLDT |
| | | | YQ |
| 14 | Gsdmdc1 | GSDMD_NTER | MPSAFEKVVKNVIKEVSGSRGDLIPVDSLRNSTSFRPYC |
| | | | LLNRKFSSSRFWKPRYSCVNLSIKDILEPSAPEPEPECF |
| | | | GSFKVSDVVDGNIQGRVMLSGMGEGKISGGAAVSDSSSA |
| | | | SMNVCILRVTQKTWETMQHERHLQQPENKILQQLRSRGD |
| | | | DLFVVTEVLQTKEEVQITEVHSQEGSGQFTLPGALCLKG |
| | | | EGKGHQSRKKMVTIPAGSILAFRVAQLLIGSKWDILLVS |
| | | | DEKQRTFEPSSGDRKAVGQRHHGLNVLAALCSIGKQLSL |
| | | | LSD |
| 15 | Gsdmdc1 | GSDMD_NTER_ | MPSAFEKVVKNVIKEVSGSRGDLIPVDSLRNSTSFRPYC |
| | | FLAG | LLNRKFSSSRFWKPRYSCVNLSIKDILEPSAPEPEPECF |
| | | | GSFKVSDVVDGNIQGRVMLSGMGEGKISGGAAVSDSSSA |
| | | | SMNVCILRVTQKTWETMQHERHLQQPENKILQQLRSRGD |
| | | | DLFVVTEVLQTKEEVQITEVHSQEGSGQFTLPGALCLKG |
| | | | EGKGHQSRKKMVTIPAGSILAFRVAQLLIGSKWDILLVS |
| | | | DEKQRTFEPSSGDRKAVGQRHHGLNVLAALCSIGKQLSL |
| | | | LSDDYKDDDDK |
| 16 | Mlkl | MLKL_FLAG_ | MDKLGQIIKLGQLIYEQCEKMKYCRKQCQRLGNRVHGLL |
| | | MT | FQPLQRLQAQGKKNLPDDITAALGRFDEVLKEANQQIEK |
| | | | SKKSHIWKFVSVGNDKILFHEVNEKLRDVWEELLLLLQV |
| | | | YHWNTVSDVSQPASWQQEDRQDAEEDGNENMKVILMQLQ |
| | | | ISVEEINKTLKQCSLKPTQEIPQDLQIKEIPKEHLGPPW |
| | | | TKLKTSKMSTIYRGEYHRSPVTIKVFNNPQAESVGIVRF |
| | | | TFNDEIKTMKKFDSPNILRIFGICIDQTVKPPEFSIVME |
| | | | YCELGTLRELLDREKDLTMSVRSLLVLRAARGLYRLHHS |
| | | | ETLHRNISSSSFLVAGGYQVKLAGFELSKTANSISRTAK |
| | | | STKAERSSSTIYVSPERLKNPFCLYDIKAEIYSFGIVLW |
| | | | EIATGKIPFEGCDSKKIRELVAEDKKQEPVGQDCPELLR |
| | | | EIINECRAHEPSQRPSVDGILERLSAVEESTDKKVDYKD |
| | | | DDDK |
| 17 | Il1b | IL1B_WT | MNNRWILHAAFLLCFSTTALSVPIRQLHYRLRDEQQKSL |
| | | | VLSDPYELKALHLNGQNINQQVIFSMSFVQGEPSNDKIP |
| | | | VALGLKGKNLYLSCVMKDGTPTLQLESVDPKQYPKKKME |
| | | | KRFVFNKIEVKSKVEFESAEFPNWYISTSQAEHKPVFLG |
| | | | NNSGQDIIDFTMESVSS |
| 18 | Ifnb | IFNB_WT | MNNRWILHAAFLLCFSTTALSINYKQLQLQERTNIRKCQ |
| | | | ELLEQLNGKINLTYRADFKIPMEMTEKMQKSYTAFAIQE |
| | | | MLQNVFLVFRNNFSSTGWNETIVVRLLDELHQQTVFLKT |
| | | | VLEEKQEERLTWEMSSTALHLKSYYWRVQRYLKLMKYNS |
| | | | YAWMVVRAEIFRNFLIIRRLTRNFQN |
| 19 | Ddx58 | FLAG_RIGI_ | MDYKDDDDKTAEQRQNLQAFRDYIKKILDPTYILSYMSS |
| | | NTER | WLEDEEVQYIQAEKNNKGPMEAASLFLQYLLKLQSEGWF |
| | | | QAFLDALYHAGYCGLCEAIESWDFQKIEKLEEHRLLLRR |
| | | | LEPEFKATVDPNDILSELSECLINQECEEIRQIRDTKGR |
| | | | MAGAEKMAECLIRSDKENWPKVLQLALEKDNSKFSELWI |
| | | | NVDKGFKRAESKADEDDGAEASSIQIFIQEEPECQNLSQ |
| | | | PGPPSEASSNNLHSPLKPRNYQLELALPAKKGKNTIICA |
| | | | PTGCGKTFVSLLICEHHLK |
| 20 | Reps1 | Reps1 | MKKVVVNGRVLELFRAAQLANDVVLQIMELCGATRLGYF |
| | | | GR |
| 21 | Il1b | IL1B_FL | MATVPELNCEMPPFDSDENDLFFEVDGPQKMKGCFQTFD |
| | | | LGCPDESIQLQISQQHINKSFRQAVSLIVAVEKLWQLPV |
| | | | SFPWTFQDEDMSTFFSFIFEEEPILCDSWDDDDNLLVCD |
| | | | VPIRQLHYRLRDEQQKSLVLSDPYELKALHLNGQNINQQ |
| | | | VIFSMSFVQGEPSNDKIPVALGLKGKNLYLSCVMKDGTP |
| | | | TLQLESVDPKQYPKKKMEKRFVFNKIEVKSKVEFESAEF |
| | | | PNWYISTSQAEHKPVFLGNNSGQDIIDFTMESVSS |
| 22 | Il1b | IL1B_WT | MNNRWILHAAFLLCFSTTALSVPIRQLHYRLRDEQQKSL |
| | | | VLSDPYELKALHLNGQNINQQVIFSMSFVQGEPSNDKIP |
| | | | VALGLKGKNLYLSCVMKDGTPTLQLESVDPKQYPKKKME |
| | | | KRFVFNKIEVKSKVEFESAEFPNWYISTSQAEHKPVFLG |
| | | | NNSGQDIIDFTMESVSS |
| 23 | n.a. | polyepitope | MAEAGQSLVISASIIVFNLLELEGDYRDDHIFSLYFMAA |
| | | vaccine | AKVVVNGRVLELFRAAQLANDVVLQIMELCGATRLGAAA |
| | | | DIPTGIPVHLELASMTNMELMSSIVHQQVFPTVASAAAG |
| | | | ISSAESLKISQAVHAAHAEINEAGREVVGSAEAGAAALL |
| | | | PDEVSGLEQLESIINFEKLTEWTSSNVMEERKI |
| 24 | Csf2 | CSF2 | MWLQNLLFLGIVVYSLSAPTRSPITVTRPWKHVEAIKEA |
| | | | LNLLDDMPVTLNEEVEVVSNEFSFKKLTCVQTRLKIFEQ |
| | | | GLRGNFTKLKGALNMTASYYQTYCPPTPETDCETQVTTY |
| | | | ADFIDSLKTFLTDIPFECKKPGQK |
| 25 | Dpagt1 | Dpagt1 | EAGQSLVISASIIVFNLLELEGDYRDDHIFSLYFM |
| 26 | Reps1 | Reps1 | KVVVNGRVLELFRAAQLANDVVLQIMELCGATRLG |
| 27 | Adpgk | Adpgk | DIPTGIPVHLELASMTNMELMSSIVHQQVFPTVAS |
| 28 | n.a. | OT-II | GISSAESLKISQAVHAAHAEINEAGREVVGSAEAG |
| 29 | n.a. | OT-I | LLPDEVSGLEQLESIINFEKLTEWTSSNVMEERKI |
| 30 | n.a. | spacer | AAA |
| 31 | Pycard | dCARD | MGSTARTGHFVDQHRQALIARVTEVDGVLDALHGSVLTE |
| | | | GQYQAVRAETTSQDKMRKLFSFVPSWNLTCKDSLLQALK |
| | | | EIHPYLVMDLEQSGGGGSGVQVETISPGDGRTFPKRGQT |
| | | | CVVHYTGMLEDGKKMDSSRDRNKPFKFMLGKQEVIRGWE |
| | | | EGVAQMSVGQRAKLTISPDYAYGATGHPGIIPPHATLVF |
| | | | DVELLKLETRGVQVETISPGDGRTFPKRGQTCVVHYTGM |
| | | | LEDGKKMDSSRDRNKPFKFMLGKQEVIRGWEEGVAQMSV |
| | | | GQRAKLTISPDYAYGATGHPGIIPPHATLVFDVELLKLE |
| 32 | Gsdmdc1 | GSDMD_WT_ | MPSAFEKVVKNVIKEVSGSRGDLIPVDSLRNSTSFRPYC |
| | | FLAG | LLNRKFSSSRFWKPRYSCVNLSIKDILEPSAPEPEPECF |
| | | | GSFKVSDVVDGNIQGRVMLSGMGEGKISGGAAVSDSSSA |
| | | | SMNVCILRVTQKTWETMQHERHLQQPENKILQQLRSRGD |
| | | | DLFVVTEVLQTKEEVQITEVHSQEGSGQFTLPGALCLKG |
| | | | EGKGHQSRKKMVTIPAGSILAFRVAQLLIGSKWDILLVS |
| | | | DEKQRTFEPSSGDRKAVGQRHHGLNVLAALCSIGKQLSL |
| | | | LSDGIDEEELIEAADFQGLYAEVKACSSELESLEMELRQ |
| | | | QILVNIGKILQDQPSMEALEASLGQGLCSGGQVEPLDGP |
| | | | AGCILECLVLDSGELVPELAAPIFYLLGALAVLSETQQQ |
| | | | LLAKALETTVLSKQLELVKHVLEQSTPWQEQSSVSLPTV |
| | | | LLGDCWDEKNPTWVLLEECGLRLQVESPQVHWEPTSLIP |
| | | | TSALYASLFLLSSLGQKPCDYKDDDDK |
| 33 | IL1b | IL1B_CALRSP | MGLLSVPLLLGLLGLAAADPAVPIRQLHYRLRDEQQKSL |
| | | | VLSDPYELKALHLNGQNINQQVIFSMSFVQGEPSNDKIP |
| | | | VALGLKGKNLYLSCVMKDGTPTLQLESVDPKQYPKKKME |
| | | | KRFVFNKIEVKSKVEFESAEFPNWYISTSQAEHKPVFLG |
| | | | NNSGQDIIDFTMESVSS |
| 34 | IL1b | IL18_WT | MNNRWILHAAFLLCFSTTALSNFGRLHCTTAVIRNINDQ |
| | | | VLFVDKRQPVFEDMTDIDQSASEPQTRLIIYMYKDSEVR |
| | | | GLAVTLSVKDSKMSTLSCKNKIISFEEMDPPENIDDIQS |
| | | | DLIFFQKRVPGHNKMEFESSLYEGHFLACQKEDDAFKLI |
| | | | LKKKDENGDKSVMFTLTNLHQS |
| 35 | CASP1 | hCASP1_RV2 | MGNLSLPTTEEFEDDAIKKAHIEKDFIAFCSSTPDNVSW |
| | | | RHPTMGSVFIGRLIEHMQEYACSCDVEEIFRKVRFSFEQ |
| | | | PDGRAQMPTTERVTLTRCFYLFPGHAYVHDAPVRDQTSG |
| | | | NYLNMQDSQGVLSSFPAPQAVQDNPAMPTSSGSEGNVKL |
| | | | CSLEEAQRIWKQKSAEIYPIMDKSSRTRLALIICNEEFD |
| | | | SIPRRTGAEVDITGMTMLLQNLGYSVDVKKNLTASDMTT |
| | | | ELEAFAHRPEHKTSDSTFLVFMSHGIREGICGKKHSEQV |
| | | | PDILQLNAIFNMLNTKNCPSLKDKPKVIIIQACRGDSPG |
| | | | VVWFKD |
| 36 | IL1B | hIL1B_SP | MTNKCLLQIALLLCFSTTALSAPVRSLNCTLRDSQQKSL |
| | | | VMSGPYELKALHLQGQDMEQQVVFSMSFVQGEESNDKIP |
| | | | VALGLKEKNLYLSCVLKDDKPTLQLESVDPKNYPKKKME |
| | | | KRFVFNKIEINNKLEFESAQFPNWYISTSQAENMPVFLG |
| | | | GTKGGQDITDFTMQFVSS |
| 37 | IL1B | hIL1B_FL | MAEVPKLASEMMAYYSGNEDDLFFEADGPKQMKCSFQDL |
| | | | DLCPLDGGIQLRISDHHYSKGFRQAASVVVAMDKLRKML |
| | | | VPCPQTFQENDLSTFFPFIFEEEPIFFDTWDNEAYVHDA |
| | | | PVRSLNCTLRDSQQKSLVMSGPYELKALHLQGQDMEQQV |
| | | | VFSMSFVQGEESNDKIPVALGLKEKNLYLSCVLKDDKPT |
| | | | LQLESVDPKNYPKKKMEKRFVFNKIEINNKLEFESAQFP |
| | | | NWYISTSQAENMPVFLGGTKGGQDITDFTMQFVSS |
| 38 | Casp1 | CASP1_RV2_ | MGIKKAHIEKDFIAFCSSTPDNVSWRHPVRGSLFIESLI |
| | | NTR | KHMKEYAWSCDLEDIFRKVRFSFEQPEFRLQMPTADRVT |
| | | | LTKRFYLFPGHLLVCDVPTRAPSAETFVATEDSKGGHPS |
| | | | SSETKEEQNKEDGTFPGLTGTLKFCPLEKAQKLWKENPS |
| | | | EIYPIMNTTTRTRLALIICNTEFQHLSPRVGAQVDLREM |
| | | | KLLLEDLGYTVKVKENLTALEMVKEVKEFAACPEHKTSD |
| | | | STFLVFMSHGIQEGICGTTYSNEVSDILKVDTIFQMMNT |
| | | | LKCPSLKDKPKVIIIQACRGEKQGVVLLKD |
| 39 | Casp1 | CASP1_RV2_ | MSEEDFLTDAIFEDDGIKKAHIEKDFIAFCSSTPDNVSW |
| | | C305G | RHPVRGSLFIESLIKHMKEYAWSCDLEDIFRKVRFSFEQ |
| | | | PEFRLQMPTADRVTLTKRFYLFPGHLLVCDVPTRAPSAE |
| | | | TFVATEDSKGGHPSSSETKEEQNKEDGTFPGLTGTLKFC |
| | | | PLEKAQKLWKENPSEIYPIMNTTTRTRLALIICNTEFQH |
| | | | LSPRVGAQVDLREMKLLLEDLGYTVKVKENLTALEMVKE |
| | | | VKEFAACPEHKTSDSTFLVFMSHGIQEGICGTTYSNEVS |
| | | | DILKVDTIFQMMNTLKCPSLKDKPKVIIIQAGRGEKQGV |
| | | | VLLKD |
| 40 | Casp1 | CASP1_RV2_ | MSEEDFLTDAIFEDDGIKKAHIEKDFIAFCSSTPDNVSW |
| | | D108A | RHPVRGSLFIESLIKHMKEYAWSCDLEDIFRKVRFSFEQ |
| | | | PEFRLQMPTADRVTLTKRFYLFPGHLLVCAVPTRAPSAE |
| | | | TFVATEDSKGGHPSSSETKEEQNKEDGTFPGLTGTLKFC |
| | | | PLEKAQKLWKENPSEIYPIMNTTTRTRLALIICNTEFQH |
| | | | LSPRVGAQVDLREMKLLLEDLGYTVKVKENLTALEMVKE |
| | | | VKEFAACPEHKTSDSTFLVFMSHGIQEGICGTTYSNEVS |
| | | | DILKVDTIFQMMNTLKCPSLKDKPKVIIIQACRGEKQGV |
| | | | VLLKD |
| 41 | Casp1 | CASP1_RV2_ | MSEEDFLTDAIFEDDGIKKAHIEKDFIAFCSSTPDNVSW |
| | | D124A | RHPVRGSLFIESLIKHMKEYAWSCDLEDIFRKVRFSFEQ |
| | | | PEFRLQMPTADRVTLTKRFYLFPGHLLVCDVPTRAPSAE |
| | | | TFVATEASKGGHPSSSETKEEQNKEDGTFPGLTGTLKFC |
| | | | PLEKAQKLWKENPSEIYPIMNTTTRTRLALIICNTEFQH |
| | | | LSPRVGAQVDLREMKLLLEDLGYTVKVKENLTALEMVKE |
| | | | VKEFAACPEHKTSDSTFLVFMSHGIQEGICGTTYSNEVS |
| | | | DILKVDTIFQMMNTLKCPSLKDKPKVIIIQACRGEKQGV |
| | | | VLLKD |
| 42 | Casp1 | CASP1_RV2_ | MSEEDFLTDAIFEDDGIKKAHIEKDFIAFCSSTPDNVSW |
| | | D143A | RHPVRGSLFIESLIKHMKEYAWSCDLEDIFRKVRFSFEQ |
| | | | PEFRLQMPTADRVTLTKRFYLFPGHLLVCDVPTRAPSAE |
| | | | TFVATEDSKGGHPSSSETKEEQNKEAGTFPGLTGTLKFC |
| | | | PLEKAQKLWKENPSEIYPIMNTTTRTRLALIICNTEFQH |
| | | | LSPRVGAQVDLREMKLLLEDLGYTVKVKENLTALEMVKE |
| | | | VKEFAACPEHKTSDSTFLVFMSHGIQEGICGTTYSNEVS |
| | | | DILKVDTIFQMMNTLKCPSLKDKPKVIIIQACRGEKQGV |
| | | | VLLKD |
| 43 | Casp1 | CASP1_RV2_ | MSEEDFLTDAIFEDDGIKKAHIEKDFIAFCSSTPDNVSW |
| | | D108A_ | RHPVRGSLFIESLIKHMKEYAWSCDLEDIFRKVRFSFEQ |
| | | D124A_ | PEFRLQMPTADRVTLTKRFYLFPGHLLVCAVPTRAPSAE |
| | | D143A | TFVATEASKGGHPSSSETKEEQNKEAGTFPGLTGTLKFC |
| | | | PLEKAQKLWKENPSEIYPIMNTTTRTRLALIICNTEFQH |
| | | | LSPRVGAQVDLREMKLLLEDLGYTVKVKENLTALEMVKE |
| | | | VKEFAACPEHKTSDSTFLVFMSHGIQEGICGTTYSNEVS |
| | | | DILKVDTIFQMMNTLKCPSLKDKPKVIIIQACRGEKQGV |
| | | | VLLKD |
| 44 | CASP1 | hCASP1_RV2_ | MGNLSLPTTEEFEDDAIKKAHIEKDFIAFCSSTPDNVSW |
| | | C306G | RHPTMGSVFIGRLIEHMQEYACSCDVEEIFRKVRFSFEQ |
| | | | PDGRAQMPTTERVTLTRCFYLFPGHAYVHDAPVRDQTSG |
| | | | NYLNMQDSQGVLSSFPAPQAVQDNPAMPTSSGSEGNVKL |
| | | | CSLEEAQRIWKQKSAEIYPIMDKSSRTRLALIICNEEFD |
| | | | SIPRRTGAEVDITGMTMLLQNLGYSVDVKKNLTASDMTT |
| | | | ELEAFAHRPEHKTSDSTFLVFMSHGIREGICGKKHSEQV |
| | | | PDILQLNAIFNMLNTKNCPSLKDKPKVIIIQAGRGDSPG |
| | | | VVWFKD |
| 45 | CASP1 | hCASP1_RV2_ | MGNLSLPTTEEFEDDAIKKAHIEKDFIAFCSSTPDNVSW |
| | | D108A | RHPTMGSVFIGRLIEHMQEYACSCDVEEIFRKVRFSFEQ |
| | | | PDGRAQMPTTERVTLTRCFYLFPGHAYVHAAPVRDQTSG |
| | | | NYLNMQDSQGVLSSFPAPQAVQDNPAMPTSSGSEGNVKL |
| | | | CSLEEAQRIWKQKSAEIYPIMDKSSRTRLALIICNEEFD |
| | | | SIPRRTGAEVDITGMTMLLQNLGYSVDVKKNLTASDMTT |
| | | | ELEAFAHRPEHKTSDSTFLVFMSHGIREGICGKKHSEQV |
| | | | GPDILQLNAIFNMLNTKNCPSLKDKPKVIIIQACRGDSP |
| | | | VVWFKD |
| 46 | CASP1 | hCASP1_RV2_ | MGNLSLPTTEEFEDDAIKKAHIEKDFIAFCSSTPDNVSW |
| | | D140A | RHPTMGSVFIGRLIEHMQEYACSCDVEEIFRKVRFSFEQ |
| | | | PDGRAQMPTTERVTLTRCFYLFPGHAYVHDAPVRDQTSG |
| | | | NYLNMQDSQGVLSSFPAPQAVQANPAMPTSSGSEGNVKL |
| | | | CSLEEAQRIWKQKSAEIYPIMDKSSRTRLALIICNEEFD |
| | | | SIPRRTGAEVDITGMTMLLQNLGYSVDVKKNLTASDMTT |
| | | | ELEAFAHRPEHKTSDSTFLVFMSHGIREGICGKKHSEQV |
| | | | PDILQLNAIFNMLNTKNCPSLKDKPKVIIIQACRGDSPG |
| | | | VVWFKD |
| 47 | CASP1 | hCASP1_RV2_ | MGNLSLPTTEEFEDDAIKKAHIEKDFIAFCSSTPDNVSW |
| | | D124A | RHPTMGSVFIGRLIEHMQEYACSCDVEEIFRKVRFSFEQ |
| | | | PDGRAQMPTTERVTLTRCFYLFPGHAYVHDAPVRDQTSG |
| | | | NYLNMQASQGVLSSFPAPQAVQDNPAMPTSSGSEGNVKL |
| | | | CSLEEAQRIWKQKSAEIYPIMDKSSRTRLALIICNEEFD |
| | | | SIPRRTGAEVDITGMTMLLQNLGYSVDVKKNLTASDMTT |
| | | | ELEAFAHRPEHKTSDSTFLVFMSHGIREGICGKKHSEQV |
| | | | PDILQLNAIFNMLNTKNCPSLKDKPKVIIIQACRGDSPG |
| | | | VVWFKD |
| 48 | CASP1 | hCASP1_RV2_ | MEEFEDDAIKKAHIEKDFIAFCSSTPDNVSWRHPTMGSV |
| | | NTR2 | FIGRLIEHMQEYACSCDVEEIFRKVRFSFEQPDGRAQMP |
| | | | TTERVTLTRCFYLFPGHAYVHDAPVRDQTSGNYLNMQDS |
| | | | QGVLSSFPAPQAVQDNPAMPTSSGSEGNVKLCSLEEAQR |
| | | | IWKQKSAEIYPIMDKSSRTRLALIICNEEFDSIPRRTGA |
| | | | EVDITGMTMLLQNLGYSVDVKKNLTASDMTTELEAFAHR |
| | | | PEHKTSDSTFLVFMSHGIREGICGKKHSEQVPDILQLNA |
| | | | IFNMLNTKNCPSLKDKPKVIIIQACRGDSPGVVWFKD |
| 49 | CASP1 | hCASP1_RV2_ | MAIKKAHIEKDFIAFCSSTPDNVSWRHPTMGSVFIGRLI |
| | | NTR | EHMQEYACSCDVEEIFRKVRFSFEQPDGRAQMPTTERVT |
| | | | LTRCFYLFPGHAYVHDAPVRDQTSGNYLNMQDSQGVLSS |
| | | | FPAPQAVQDNPAMPTSSGSEGNVKLCSLEEAQRIWKQKS |
| | | | AEIYPIMDKSSRTRLALIICNEEFDSIPRRTGAEVDITG |
| | | | MTMLLQNLGYSVDVKKNLTASDMTTELEAFAHRPEHKTS |
| | | | DSTFLVFMSHGIREGICGKKHSEQVPDILQLNAIFNMLN |
| | | | TKNCPSLKDKPKVIIIQACRGDSPGVVWFKD |
| 50 | CASP1 | hCASP1_RV2_ | MAIKKAHIEKDFIAFCSSTPDNVSWRHPTMGSVFIGRLI |
| | | NTR_GSL | EHMQEYACSCDVEEIFRKVRFSFEQPDGRAQMPTTERVT |
| | | | LTRCFYLFPGHGSGSGSGSGNPAMPTSSGSEGNVKLCSL |
| | | | EEAQRIWKQKSAEIYPIMDKSSRTRLALIICNEEFDSIP |
| | | | RRTGAEVDITGMTMLLQNLGYSVDVKKNLTASDMTTELE |
| | | | AFAHRPEHKTSDSTFLVFMSHGIREGICGKKHSEQVPDI |
| | | | LQLNAIFNMLNTKNCPSLKDKPKVIIIQACRGDSPGVVW |
| | | | FKD |
| 51 | CASP1 | hCASP1_RV2_ | MAIKKAHIEKDFIAFCSSTPDNVSWRHPTMGSVFIGRLI |
| | | NTR_CCS | EHMQEYACSCDVEEIFRKVRFSFEQPDGRAQMPTTERVT |
| | | | LTRCFYLFPGHAYVHDAPVRNPAMPTSSGSEGNVKLCSL |
| | | | EEAQRIWKQKSAEIYPIMDKSSRTRLALIICNEEFDSIP |
| | | | RRTGAEVDITGMTMLLQNLGYSVDVKKNLTASDMTTELE |
| | | | AFAHRPEHKTSDSTFLVFMSHGIREGICGKKHSEQVPDI |
| | | | LQLNAIFNMLNTKNCPSLKDKPKVIIIQACRGDSPGVVW |
| | | | FKD |
| 52 | CASP1 | hCASP1_WT | MADKVLKEKRKLFIRSMGEGTINGLLDELLQTRVLNKEE |
| | | | MEKVKRENATVMDKTRALIDSVIPKGAQACQICITYICE |
| | | | EDSYLAGTLGLSADQTSGNYLNMQDSQGVLSSFPAPQAV |
| | | | QDNPAMPTSSGSEGNVKLCSLEEAQRIWKQKSAEIYPIM |
| | | | DKSSRTRLALIICNEEFDSIPRRTGAEVDITGMTMLLQN |
| | | | LGYSVDVKKNLTASDMTTELEAFAHRPEHKTSDSTFLVF |
| | | | MSHGIREGICGKKHSEQVPDILQLNAIFNMLNTKNCPSL |
| | | | KDKPKVIIIQACRGDSPGVVWFKDSVGVSGNLSLPTTEE |
| | | | FEDDAIKKAHIEKDFIAFCSSTPDNVSWRHPTMGSVFIG |
| | | | ERLIEHMQEYACSCDVEEIFRKVRFSFEQPDGRAQMPTT |
| | | | RVTLTRCFYLFPGH |
| 53 | GSDMD | hGSDMD_WT_ | MGSAFERVVRRVVQELDHGGEFIPVTSLQSSTGFQPYCL |
| | | FLAG | VVRKPSSSWFWKPRYKCVNLSIKDILEPDAAEPDVQRGR |
| | | | SFHFYDAMDGQIQGSVELAAPGQAKIAGGAAVSDSSSTS |
| | | | MNVYSLSVDPNTWQTLLHERHLRQPEHKVLQQLRSRGDN |
| | | | VYVVTEVLQTQKEVEVTRTHKREGSGRFSLPGATCLQGE |
| | | | GQGHLSQKKTVTIPSGSTLAFRVAQLVIDSDLDVLLFPD |
| | | | KKQRTFQPPATGHKRSTSEGAWPQLPSGLSMMRCLHNFL |
| | | | TDGVPAEGAFTEDFQGLRAEVETISKELELLDRELCQLL |
| | | | LEGLEGVLRDQLALRALEEALEQGQSLGPVEPLDGPAGA |
| | | | VLECLVLSSGMLVPELAIPVVYLLGALTMLSETQHKLLA |
| | | | EALESQTLLGPLELVGSLLEQSAPWQERSTMSLPPGLLG |
| | | | NSWGEGAPAWVLLDECGLELGEDTPHVCWEPQAQGRMCA |
| | | | LYASLALLSGLSQEPHDYKDDDDK |
| 54 | CASP1 | hCASP1_ | MADKVLKEKRKLFIRSMGEGTINGLLDELLQTRVLNKEE |
| | | CARD | MEKVKRENATVMDKTRALIDSVIPKGAQACQICITYICE |
| | | | EDSYLAGTLGLSAD |
| 55 | CASP1 | hCASP1_ | QTSGNYLNMQDSQGVLSSFPAPQAVQD |
| | | CDL | |
| 56 | CASP1 | hCASP1_ | NPAMPTSSGSEGNVKLCSLEEAQRIWKQKSAEIYPIMDK |
| | | p20 | SSRTRLALIICNEEFDSIPRRTGAEVDITGMTMLLQNLG |
| | | | YSVDVKKNLTASDMITELEAFAHRPEHKTSDSTFLVFMS |
| | | | HGIREGICGKKHSEQVPDILQLNAIFNMLNTKNCPSLKD |
| | | | KPKVIIIQACRGDSPGVVWFKD |
| 57 | CASP1 | hCASP1_ | SVGVSGNLSLPTTEEFEDD |
| | | IDL | |
| 58 | CASP1 | hCASP1_ | AIKKAHIEKDFIAFCSSTPDNVSWRHPTMGSVFIGRLIE |
| | | p10 | HMQEYACSCDVEEIFRKVRFSFEQPDGRAQMPTTERVTL |
| | | | TRCFYLFPGH |

TABLE 3
Overview of co-stimulatory molecules.

| Nr. | Type | Name | Human protein name |
|---|---|---|---|
| 1 | cytokine | CSF1 | Macrophage colony- |
| | | | stimulating factor 1 |
| 2 | cytokine | CSF2 | Granulocyte-macrophage |
| | | | colony-stimulating factor |
| 3 | cytokine | TNF | Tumor necrosis factor |
| 4 | cytokine | IFNβ | Interferon beta |
| 5 | cytokine | IFNγ | Interferon gamma |
| 6 | cytokine | Flt3L | Fms-related tyrosine |
| | | | kinase 3 ligand |
| 7 | cytokine | IL1β | Interleukin-1 beta |
| 8 | cytokine | IL2 | Interleukin-2 |
| 9 | cytokine | IL4 | Interleukin-4 |
| 10 | cytokine | IL6 | Interleukin-6 |
| 11 | cytokine | IL7 | Interleukin-7 |
| 12 | cytokine | IL10 | Interleukin-10 |
| 13 | cytokine | IL12 | Interleukin-12 |
| 14 | cytokine | IL15 | Interleukin-15 |
| 15 | cytokine | IL18 | Interleukin-18 |
| 16 | cytokine | IL21 | Interleukin-21 |
| 17 | cytokine | IL23 | Interleukin-23 |
| 18 | cytokine | IL27 | Interleukin-27 |
| 19 | cytokine | IL35 | Interleukin-35 |
| 20 | chemokine | MIP1α | C-C motif chemokine 3 |
| 21 | chemokine | MIP1β | C-C motif chemokine 4 |
| 22 | chemokine | MIP3α | C-C motif chemokine 20 |
| 23 | chemokine | MIP3β | C-C motif chemokine 19 |
| 24 | chemokine | RANTES | C-C motif chemokine 5 |
| 25 | chemokine | MCP-1 | C-C motif chemokine 2 |
| 26 | chemokine | MCP-2 | C-C motif chemokine 8 |
| 27 | chemokine | MCP-3 | C-C motif chemokine 7 |
| 28 | chemokine | MCP-4 | C-C motif chemokine 13 |
| 29 | chemokine | GCP-2 | C-X-C motif chemokine 6 |
| 30 | chemokine | NAP-2 | Platelet basic protein |
| 31 | chemokine | IL-8 | Interleukin-8 |
| 33 | transmembrane protein | CD40L | CD40 ligand |
| | (soluble version) | | |
| 34 | transmembrane protein | OX40 | Tumor necrosis factor receptor |
| | (soluble version) | | superfamily member 4 |

TABLE 4
Caspase-1 constructs. For a detailed description
of caspase-1 constructs, also see FIG. 6.

| | | | |
|---|---|---|---|
| | | | SEQ |
| Gene | | | ID |
| name | Construct | Modification | NO |

Mus musculus

| | | | |
|---|---|---|---|
| Casp 1 | CASP1_RV2_C305G | CASP1_RV2, active site | 39 |
| | | mutant | |
| | CASP1_RV2_NTR | CASP1_RV2, no extension | 38 |
| | | N-terminal to p10 | |

Homo sapiens

| | | | |
|---|---|---|---|
| CASP1 | hCASP1_WT | wild-type (wt) | 52 |
| | hCASP1_RV2_C305G | hCASP1_RV2, active site | 44 |
| | | mutant | |
| | hCASP1_RV2_NTR2 | hCASP1_RV2, small (6 AA) | 48 |
| | | N-terminal IDL extension | |
| | hCASP1_RV2_NTR | hCASP1_RV2, no N-terminal | 49 |
| | | IDL extension | |
| | hCASP1_RV2_NTR_CCS | hCASP1_RV2_NTR, short | 51 |
| | | p10-p20 linker AYVHDAPVR | |
| | hCASP1_RV2_NTR_GSL | hCASP1_RV2_NTR, short | 50 |
| | | p10-p20 linker GSGSGSGSG | |
| | hGSDMD_WT_FLAG | wild-type, FLAG-tagged | 53 |

1. An immune-stimulating composition comprising a pharmacologically acceptable excipient and an expression construct encoding a constitutively active pro-inflammatory caspase-1 which comprises swapped p10 and p20 domains.

2. The immune-stimulating composition according to claim 1, wherein the constitutively active pro-inflammatory caspase-1 lacks a caspase-recruitment domain (CARD).

3. The immune-stimulating composition according to claim 1, wherein the constitutively active pro-inflammatory caspase-1 is one in which a glycine corresponding to G403 (SEQ ID NO: 52) is located at a distance from 0 to 40 amino acids residues from a cysteine corresponding to C136 (SEQ ID NO: 52).

4. The immune-stimulating composition according to claim 1, wherein the constitutively active pro-inflammatory caspase-1 lacks a p20-p10 interdomain linker (IDL).

5. The immune-stimulating composition according to claim 1, wherein the constitutively active pro-inflammatory caspase-1 is a human caspase-1.

6. The immune-stimulating composition according to claim 1, wherein the swapped p10 and p20 domains are connected by a protease cleavable site.

7. The immune-stimulating composition according to claim 1, further comprising at least one antigen or at least one RNA or DNA nucleic acid sequence encoding an antigen.

8. The immune-stimulating composition according to claim 1, further comprising a genetic vaccine.

9. The immune-stimulating composition according to claim 1, wherein said expression construct is a non-viral expression construct.

10. The immune-stimulating composition according to claim 1, further comprising: (i) an accessory immune stimulating molecule, (ii) an immune stimulating molecule, or a combination of (i) and (ii).

11. A method of stimulating an immune response in an individual in need thereof, comprising administering to the individual the immune-stimulating composition of claim 1.

12. The method according to claim 11, wherein said immune response is directed against a tumor or infection that is present in the individual.

13. The method according to claim 11, wherein said immune-stimulating composition is administered into a tumor of the individual.

14. The method according to claim 11, wherein said immune-stimulating composition is combined with an expression construct coding for another immune stimulating molecule.

15. The method according to claim 11, wherein said wherein said immune-stimulating composition is administered in combination with one or more accessory molecules, a further immune stimulating molecule, or a combination thereof.

16. The method according to claim 11, wherein said one or more accessory molecules are selected from the group consisting of macrophage colony-stimulating factor 1, granulocyte-macrophage colony-stimulating factor, tumor necrosis factor, interferon beta, interferon gamma, Fms-related tyrosine kinase 3 ligand, interleukin-1 beta, interleukin-2, interleukin-4, interleukin-6, interleukin-7, interleukin-10, interleukin-12, interleukin-15, interleukin-18, interleukin-21, interleukin-23, interleukin-27, interleukin-35, C-C motif chemokine 3, C-C motif chemokine 4, C-C motif chemokine 20, C-C motif chemokine 19, C-C motif chemokine 5, C-C motif chemokine 2, C-C motif chemokine 8, C-C motif chemokine 7, C-C motif chemokine 13, C-X-C motif chemokine 6, platelet basic protein, interleukin-8, CD40 ligand and tumor necrosis factor receptor superfamily member 4.

17. The method according to claim 15, wherein said one or more accessory molecules are interleukin-12 (IL-12), granulocyte-macrophage colony-stimulating factor (CSF2), or a combination thereof.

18. The method according to claim 11, wherein the individual is a human suffering from a disease, or at risk to suffer from a disease.
